(19) United States
(12) Patent Application Publication
(10) Pub. No.: US 2018/0237836 A1
(43) Pub. Date: Aug. 23, 2018

(54) Microdroplet-Based Multiple Displacement Amplification (MDA) Methods and Related Compositions

(71) Applicant: The Regents of the University of California, Oakland, CA (US)

(72) Inventors: Adam R. Abate, Daly City, CA (US); Freeman Lan, San Francisco, CA (US); Shaun Lim, Eastview (SG); Angus Sidore, San Francisco, CA (US)

(21) Appl. No.: 15/753,132

(22) PCT Filed: Aug. 16, 2016
(86) PCT No.: PCT/US16/47199

§ 371 Date: Feb. 15, 2018

Related U.S. Application Data
(60) Provisional application No. 62/206,202, filed on Aug. 17, 2015.

(51) Int. Cl. C12Q 1/6827; C12P 19/34; C12Q 1/6844; C40B 30/06; C12N 9/12

(52) U.S. Cl. CPC C12Q 1/6827; C12P 19/34; C12Q 1/6844; C40B 30/06; C12Q 2563/159; C12Q 2525/179; C12Q 2531/119; C12Q 2535/122; C12Q 2537/16; C12N 9/1252

(57) ABSTRACT

Methods for non-specifically amplifying a nucleic acid template molecule are provided. The methods may be used to amplify nucleic acid template molecule(s) for sequencing, e.g., for sequencing the genomes of uncultivable microbes or sequencing to identify copy number variation in cancer cells. Aspects of the disclosed methods may include non-specifically amplifying a nucleic acid template molecule, including encapsulating in a microdroplet a nucleic acid template molecule obtained from a biological sample, introducing multiple displacement amplification (MDA) reagents and a plurality of MDA primers into the microdroplet, and incubating the microdroplet under conditions effective for the production of MDA amplification products, wherein the incubating is effective to produce MDA amplification products from the nucleic acid template molecule.

### CROSS-REFERENCE

This application claims the benefit of U.S. Provisional Application No. 62/206,202, filed Aug. 17, 2015, which application is incorporated herein by reference in its entirety and for all purposes.

### GOVERNMENT SUPPORT

This invention was made with government support under grant no. DBI1253293 awarded by the National Science Foundation; grant nos. HG007233, R01 EB019453 and AR068129 awarded by the National Institutes of Health; grant nos. HR0011-12-C-0065 and HR0011-12-C-0066 awarded by the Department of Defense; and grant no. N66001-12-C-4211 awarded by the Space and Naval Warfare Systems Center. The government has certain rights in the invention.

### INTRODUCTION

The ability to efficiently sequence small quantities of nucleic acid, e.g., DNA, is important for applications ranging from the assembly of uncultivable microbial genomes to the identification of cancer-associated mutations. To obtain sufficient quantities of nucleic acid for sequencing, the limited starting material must be amplified significantly. However, existing methods often yield errors or non-uniformity of coverage, reducing sequencing data quality.

Single cell sequencing is an invaluable tool in microbial ecology and has enhanced the analysis of communities ranging from the ocean (Yoon et al. (2011) “Single-cell genomics reveals organismal interactions in uncultivated marine protists.” Science, 332, 714-717) to the human mouth (Marcy et al. (2007) “Dissecting biological ‘dark matter’ with single-cell genetic analysis of rare and uncultivated TM7 microbes from the human mouth.” Proc. Natl. Acad. Sci. U.S.A, 104, 11889-11894). Because the majority of microorganisms cannot be cultured (Hutchison III, C. A. H. and Venter, J. C. (2006) “Single-cell genomics.” Nat. Biotechnol., 24, 657-658), obtaining sufficient quantities of DNA for sequencing requires significant amplification of single-cell genomes. However, existing methods for accomplishing this are prone to amplification bias, making sequencing inefficient and costly. Consequently, there has been a sustained effort to develop new methods to uniformly amplify small quantities of DNA.

On method is to modify the PCR reaction to enable non-specific amplification. Primer Extension Preamplification (PEP) and Degenerate Oligonucleotide-Primed PCR (DOP-PCR), for example, use modified primers and thermal cycling conditions to enable non-specific annealing and amplification of most DNA sequences (Zhang et al. (1992) “Whole genome amplification from a single cell: implications for genetic analysis.” Proc. Natl. Acad. Sci. U.S.A., 89, 5847-5851, Telenius et al. (1992) “Degenerate oligonucleotide-primed PCR: general amplification of target DNA by a single degenerate primer.” Genomics, 13, 718-725). However, amplification bias remains a major challenge for these methods: the products typically do not fully cover the original template and possess significant variation in coverage (Cheung, V. G. and Nelson, S. F. (1996) “Whole genome amplification using a degenerate oligonucleotide primer allows hundreds of genotypes to be performed on less than one nanogram of genomic DNA.” Proc. Natl. Acad. Sci. U.S.A, 93, 14676-14679, Dean et al. (2002) “Comprehensive human genome amplification using multiple displacement amplification.” Proc. Natl. Acad. Sci. U.S.A, 99, 5261-5266). Multiple Annealing and Looping Based Amplification Cycles (MALBAC) reduces this bias with primers that cause amplicons to self-anneal in a loop; this suppresses exponential amplification of dominant products and equalizes amplification across the templates (Zong et al. (2012) “Genome-wide detection of single-nucleotide and copy-number variations of a single human cell.” Science, 338, 1622-6). Nevertheless, the specialized polymerase required for this reaction is prone to copy errors that propagate through cycling, resulting in increased error rates (Id.).

Multiple displacement amplification (MDA) enables non-specific amplification with minimal error through the use of the highly accurate enzyme Φ29 DNA polymerase (Esteban et al. (1993) “Fidelity of phi29 DNA Polymerase.” J. Biol. Chem., 268, 2719-2726). In addition, Φ29 DNA polymerase displaces Watson-Crick base-paired strands, enabling exponential amplification of template molecules without thermally-induced denaturation (Dean et al. (2002) “Comprehensive human genome amplification using multiple displacement amplification.” Proc. Natl. Acad. Sci. U.S.A, 99, 5261-5266). Nevertheless, two major problems persist with MDA: amplification of contaminating DNA (Raghunathan et al. (2005) “Genomic DNA Amplification from a Single Bacterium Genomic DNA Amplification from a Single Bacterium.” Appl. Environ. Microbiol., 71, 3342-3347) and highly uneven amplification of single-cell genomes (Dean et al. (2001) “Rapid amplification of plasmid and phage DNA using Phi29 DNA polymerase and multiply-primed rolling circle amplification.” Genome Res., 11, 1095-1099, Hosono et al. (2003) “Unbiased whole-genome amplification directly from clinical samples.” Genome Res., 13, 954-964). These problems yield numerous challenges when sequencing MDA-amplified material, including incomplete genome assembly, gaps in genome coverage, and biased counts of replicated sequences, which are of biological relevance in a variety of applications such as assessing copy number variants in cancer. Due to its simplicity and accuracy, several strategies have been employed to reduce MDA amplification bias, including augmenting reactions with trehalose (Pan et al. (2008) “A procedure for highly specific, sensitive, and unbiased whole-genome amplification.” Proc. Natl. Acad. Sci. U.S.A, 105, 15499-15504), reducing reaction volumes (Hutchison et al. (2005) “Cell-free cloning using phi29 DNA polymerase.” Proc. Natl. Acad. Sci. U.S.A, 102, 17332-17336), and using nanoliter-scale microfluidic chambers to reduce the diversity in isolated pools (Marcy et al. (2007) “Nanoliter reactors improve multiple displacement amplification of genomes from single cells.” PLoS Genet., 3, 1702-1708, Gole et al. (2013) “Massively parallel polymerase cloning and genome sequencing of single cells using nanoliter microwells.” Nat. Biotechnol., 31, 1126-32). While these methods help to mitigate the problems associated with MDA, robust and uniform amplification of low-input material remains a challenge.

The present disclosure provides methods and related compositions which help to address the above deficiencies in the art.

### SUMMARY

Methods for non-specifically amplifying a nucleic acid template molecule are provided. In certain aspects, the methods may be used to amplify nucleic acid template molecule(s) for sequencing, e.g., for sequencing the genomes of uncultivable microbes or sequencing to identify copy number variation in cancer cells.

Methods of the present disclosure include methods for the amplification of nucleic acids, e.g., genomic DNA, from a biological sample. Using microfluidics, components of the biological sample, e.g., genomic DNA, may be encapsulated into microdroplets having an internal volume ranging between 0.001 picoliters to about 1000 picoliters in volume. The components encapsulated in each microdroplet may then be amplified and assayed as described more fully herein. In some embodiments, nucleic acid template molecules are encapsulated in microdroplets such that each microdroplet includes either zero or one nucleic acid template molecule. In other words, in some embodiments the nucleic acid template molecules are encapsulated at a ratio of one per microdroplet or less. Compartmentalizing and amplifying a very few molecules, e.g., 10 or less, 5 or less, such as a single molecule, affords a number of benefits for obtaining accurate sequence data with uniform coverage. Because the molecules are isolated from one another, each reaction progresses to saturation irrespective of when it initiates—a stochastic process that, in bulk, is the primary source of bias (Rodrigue et al. (2009) “Whole genome amplification and de novo assembly of single bacterial cells.” PLoS One, 4.). As discussed in greater detail herein, these benefits greatly enhance accuracy.

Aspects of the disclosed methods may include non-specifically amplifying a nucleic acid template molecule, including encapsulating in a microdroplet a nucleic acid template molecule obtained from a biological sample; introducing multiple displacement amplification (MDA) reagents and a plurality of MDA primers into the microdroplet; and incubating the microdroplet under conditions effective for the production of MDA amplification products, wherein the incubating is effective to produce MDA amplification products from the nucleic acid template molecule. In certain aspects, the encapsulating includes encapsulating in a plurality of microdroplets a plurality of nucleic acid template molecules obtained from one or more biological samples, the introducing includes introducing MDA reagents and a plurality of MDA primers into each of the plurality of microdroplets, and the incubating includes incubating the plurality of microdroplets under conditions effective for the production of MDA amplification products, wherein the incubating is effective to produce MDA amplification products from the nucleic acid template molecules. In certain aspects, the MDA amplification products include a single MDA amplification product or a plurality of different MDA amplification products. In certain aspects, the biological sample includes one or more cells.

Aspects of the methods may also include a method for performing copy-number variation (CNV) analysis on a population of nucleic acids isolated from a biological sample, including fragmenting the population of nucleic acids; encapsulating the fragmented population of nucleic acids in a plurality of microdroplets; introducing Multiple Displacement Amplification (MDA) reagents and a plurality of MDA primers, into each of the plurality of microdroplets; incubating the microdroplets under conditions effective for the production of MDA amplification products, wherein the incubating is effective to produce MDA amplification products from the nucleic acid template molecules; and sequencing the MDA amplification products to determine the copy number of one or more nucleic acid sequences in the population of nucleic acids. In some embodiments, a fragmenting step is not performed prior to the encapsulating step.

Certain aspects of the present disclosure may include a composition including a microdroplet, including: a single nucleic acid template molecule; and an MDA mixture, including: a polymerase enzyme capable of non-specifically amplifying the nucleic acid template molecule and a plurality of MDA primers.

In practicing the subject methods, several variations may be employed. For example, a wide range of different MDA-based assays may be employed. The number and nature of primers used in such assays may vary, based at least in part on the type of assay being performed, the nature of the biological sample, and/or other factors. In certain aspects, the number of primers that may be added to a microdroplet may be 1 to 100 or more, and/or may include primers to bind from about 1 to 100 or more different nucleic acid sequences.

The microdroplets themselves may vary, including in size, composition, contents, and the like. Microdroplets may generally have an internal volume of about 0.001 to 1000 picoliters or more. Further, microdroplets may or may not be stabilized by surfactants and/or particles.

The means by which reagents are added to a microdroplet may vary greatly. Reagents may be added in one step or in multiple steps, such as 2 or more steps, 4 or more steps, or 10 or more steps. In certain aspects, reagents may be added using techniques including droplet coalescence, picoinjection, multiple droplet coalescence, and the like, as shall be described more fully herein. In certain embodiments, reagents are added by a method in which the injection fluid itself acts as an electrode. The injection fluid may contain one or more types of dissolved electrolytes that permit it to be used as such. Where the injection fluid itself acts as the electrode, the need for metal electrodes in the microfluidic chip for the purpose of adding reagents to a droplet may be obviated. In certain embodiments, the injection fluid does not act as an electrode, but one or more liquid electrodes are utilized in place of metal electrodes.

Various ways of detecting the presence or absence of MDA products may be employed, using a variety of different detection components. Detection components of interest include, but are not limited to, fluorescein and its derivatives; rhodamine and its derivatives; cyanine and its derivatives; coumarin and its derivatives; Cascade Blue and its derivatives; Lucifer Yellow and its derivatives; BODIPY and its derivatives; and the like. Exemplary fluorophores include indocarbocyanine (C3), indodicarbocyanine (C5), Cy3, Cy3.5, Cy5, Cy5.5, Cy7, Texas Red, Pacific Blue, Oregon Green 488, Alexa fluor-355, Alexa Fluor 488, Alexa Fluor 532, Alexa Fluor 546, Alexa Fluor-555, Alexa Fluor 568, Alexa Fluor 594, Alexa Fluor 647, Alexa Fluor 660, Alexa Fluor 680, JOE, Lissamine, Rhodamine Green, BODIPY, fluorescein isothiocyanate (FITC), carboxy-fluorescein (FAM), phycoerythrin, rhodamine, dichlororhodamine (dRhodamine), carboxy tetramethylrhodamine (TAMRA), carboxy-X-rhodamine (ROX), LIZ, VIC, NED, PET, SYBR, PicoGreen, RiboGreen, and the like. Detection components may include beads (e.g., magnetic or fluorescent beads, such as Luminex beads) and the like. In certain aspects, detection may involve holding a microdroplet at a fixed position during nucleic acid amplification so that it can be repeatedly imaged. In certain aspects, detection may involve fixing and/or permeabilizing one or more cells in one or more microdroplets.

Suitable subjects for the methods disclosed herein, e.g., suitable subjects from which a biological sample may be obtained for analysis, include mammals, e.g., humans. The subject may be one that exhibits clinical presentations of a disease condition, or has been diagnosed with a disease. In certain aspects, the subject may be one that has been diagnosed with cancer, exhibits clinical presentations of cancer, or is determined to be at risk of developing cancer due to one or more factors such as family history, environmental exposure, genetic mutation(s), lifestyle (e.g., diet and/or smoking), the presence of one or more other disease conditions, and the like.

### BRIEF DESCRIPTION OF THE DRAWINGS

The invention may be best understood from the following detailed description when read in conjunction with the accompanying drawings. Included in the drawings are the following figures:

FIG. 1, Panels A-C provide an illustration of how compartmentalized MDA enhances sequencing coverage. Panel A: Amplifying nucleic acid template molecule(s) via bulk multiple displacement amplification (bulk MDA). As illustrated in Panel A, uncompartmentalized amplification does not constrain the exponential activity of Φ29 DNA Polymerase, leading to sequencing bias. Panel B: Amplifying nucleic acid template molecule(s) via shaken emulsion MDA. As illustrated in Panel B, compartmentalization of the reaction in a shaken emulsion enhances sequencing coverage; however, the polydispersity of the emulsion leads to some sequencing bias. Panel C: Amplifying nucleic acid template molecule(s) via digital droplet MDA (ddMDA). As illustrated in Panel C, compartmentalization of reaction generated using a microfluidic device yields even greater sequencing coverage due to the high uniformity of the reaction.

FIG. 2, Panels A-B provide a demonstration of digital droplet MDA and its utility for nonspecific DNA quantification. Panel A: Fluorescence microscopy images of droplets subjected to digital droplet MDA (ddMDA—upper row) and digital droplet PCR (ddPCR—lower row) for three concentrations of input material. Fluorescence was obtained using Eva Green (ddMDA) and Taqman probe (ddPCR). The disparity between digital MDA and PCR quantification corresponds to the nonspecific nature of MDA compared to specific PCR amplification. Panel B: Fraction of observed versus predicted droplets. Fraction of fluorescent droplets is predicted assuming Poisson encapsulation of whole genomes. While ddPCR encapsulates one positive droplet per genome, ddMDA encapsulates one positive droplet per DNA segment. This enables nonspecific quantitation of nucleic acids and allows for the calculation of contamination and fragmentation of the sample.

FIG. 3, Panels A-B illustrate the impact of compartmentalized amplification on coverage uniformity. Panel A: Relative coverage, defined as the number of reads for each base divided by the mean number of reads for the whole genome (Ross et al. (2013) “Characterizing and measuring bias in sequence data.” Genome Biol., 14, R51), plotted versus genome position. Relative coverage was measured for three scenarios: unamplified E. coli (top), standard bulk MDA (middle), and digital droplet MDA (bottom) and consolidated into 10 kbp bins. Panel B: Probability density as a function of relative coverage for Unamplified E. coli, Bulk MDA, and Digital Droplet MDA. While coverage distribution has negligible undercovered reads for Unamplified E. coli, Bulk MDA shows a significant fraction of bases with very low coverage, a known property of MDA. Digital droplet MDA appears as a mixture of these distributions, indicating that coverage is enhanced.

FIG. 4, Panels A-B illustrate a comparison between PicoPLEX WGA and ddMDA. Panel A: relative coverage as a function of genome position of 0.5 pg E. coli DNA amplified using the PicoPLEX WGA kit compared to 0.5 pg E. coli DNA amplified using ddMDA. Data points were consolidated into 10 kb bins. Panel B: probable density as a function of relative coverage for PicoPLEX WGA and ddMDA. As shown, PicoPlex WGA appears to have a greater proportion of bases with minimal coverage compared to ddMDA.

FIG. 5, Panels A-C illustrates relative coverage of standard bulk MDA (Panel A), shaken emulsion MDA (Panel B), and digital droplet MDA (Panel C) of E. coli DNA amplified from 5 picograms (˜1000 E. coli genomes), 0.5 picograms (˜100 E. coli genomes), and 0.05 picograms (˜10 E. coli genomes). Data points were consolidated into 10 kb bins. Two samples were excluded from the analysis: bulk MDA 3 had less than 5% of sequenced DNA aligned to the E. coli genome, while ddMDA3 was not indexed properly and thus did not yield any sequencing data.

FIG. 6, Panels A-C provide a comparison of bias for three different MDA methods for three input DNA concentrations. Plots on the right show each metric normalized to the bulk MDA measurements averaged over all three input DNA concentrations. Panel A: Dropout rate, defined as the fraction of bases covered at less than 10% the mean coverage, plotted against input DNA concentration. Panel B: Coverage spread, measured as the root mean square of the relative coverage. Panel C: Informational entropy, defined as ∫p log(1/p), where p is the probability of observing reads within defined windows of the genome.

FIG. 7, Panels A-B illustrate that ddMDA of single E. coli cells significantly enhances coverage uniformity. Panel A: Relative coverage, defined as the number of reads for each base divided by the mean number of reads for the whole genome, plotted versus genome position. Relative coverage is measured for two cells amplified via bulk MDA (first panel) and two cells amplified via ddMDA (second panel) consolidated into 10 kbp bins. Gaps in coverage plots represent complete dropout of a given 10 kbp bin. Panel B: Probability density as a function of relative coverage for two cells amplified via bulk MDA and two cells amplified via ddMDA. The two cells amplified by bulk MDA show a significant fraction of bases with very low coverage, while the cells amplified by ddMDA show much more uniform coverage.

FIG. 8, Panels A-C illustrate a comparisons of droplet size distribution between shaken emulsion MDA and ddMDA. Panel A: bright field microscopy images of representative shaken emulsion and ddMDA reactions. Panel B: normalized diameter distribution of droplets measured in micrometers. Panel C: normalized volume distribution of droplets measured in picoliters.

FIG. 9 provides an illustration and a microscopy image of an exemplary microfluidic device, which may be used in the methods described herein.

FIG. 10 provides equations for the definitions described in FIG. 4. Dropout metric represents the fraction of bases that are covered less than 10% of the mean coverage. Coverage spread is defined as the root mean square of the relative coverage. Informational entropy is defined as a sum of the product of the given probability with its base-2 logarithm.

### DETAILED DESCRIPTION

Methods for non-specifically amplifying a nucleic acid template molecule are provided. In certain aspects, the methods may be used to amplify nucleic acid template molecule(s) for sequencing, e.g., sequencing the genomes of uncultivable microbes or sequencing to identify copy number variation in cancer cells.

Before the present invention is described in greater detail, it is to be understood that this invention is not limited to particular embodiments described, as such may vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials may now be described. Any and all publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a microdroplet” includes a plurality of such microdroplets and reference to “the microdroplet” includes reference to one or more microdroplets, and so forth.

It is further noted that the claims may be drafted to exclude any element which may be optional. As such, this statement is intended to serve as antecedent basis for use of such exclusive terminology as “solely”, “only” and the like in connection with the recitation of claim elements, or the use of a “negative” limitation.

Any and all publications mentioned herein are incorporated herein by reference to disclose and describe the methods and/or materials in connection with which the publications are cited. It is understood that the present disclosure supersedes any disclosure of an incorporated publication to the extent there is a contradiction. Further, the dates of any such publications provided may be different from the actual publication dates which may need to be independently confirmed.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed. To the extent such publications may set out definitions of a term that conflict with the explicit or implicit definition of the present disclosure, the definition of the present disclosure controls.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

Although any methods and materials similar or equivalent to those described herein can be used in the practice of the present invention, some potential and exemplary methods and materials are now described.

### Methods

As summarized above, aspects of the invention include methods for the amplification of nucleic acids from biological samples. Such methods may be utilized to facilitate the sequencing and/or quantitation of one or more nucleic acid sequences, e.g., one or more nucleic acids derived from one or more cells, e.g., one or more tumor or non-tumor cells. Aspects of interest include methods of performing copy-number variation analysis on a population of nucleic acids, e.g., genomic nucleic acids isolated from a single cell, such as a tumor cell, e.g., a circulating tumor cell (CTC).

As used herein, the phrase “biological sample” encompasses a variety of sample types of biological origin which sample types contain one or more nucleic acids. For example, the definition of “biological sample” encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents, solubilization, or enrichment for certain components, such as polynucleotides. The term “biological sample” encompasses a clinical sample, and also includes cells in culture, cell supernatants, cell lysates, cells, serum, plasma, biological fluid, and tissue samples.

As described more fully herein, in various aspects the subject methods may be used to amplify nucleic acids from such biological samples. Biological samples of particular interest may include cells (e.g., circulating tumor cells).

The terms “nucleic acid”, “nucleic acid molecule”, “oligonucleotide” and “polynucleotide” are used interchangeably and refer to a polymeric form of nucleotides of any length, either deoxyribonucleotides or ribonucleotides, or analogs thereof. The terms encompass, e.g., DNA, RNA and modified forms thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, control regions, isolated RNA of any sequence, nucleic acid probes, and primers. The nucleic acid molecule may be linear or circular. Nucleic acids can have any of a variety of structural configurations, e.g., be single stranded, double stranded, or a combination of both, as well as having higher order intra- or intermolecular secondary/tertiary structures, e.g., hairpins, loops, triple stranded regions, etc.

The term “nucleic acid sequence” or “oligonucleotide sequence” refers to a contiguous string of nucleotide bases and in particular contexts also refers to the particular placement of nucleotide bases in relation to each other as they appear in a oligonucleotide.

The terms “complementary” or “complementarity” refer to polynucleotides (i.e., a sequence of nucleotides) related by base-pairing rules. For example, the sequence “5′-AGT-3′,” is complementary to the sequence “5′-ACT-3”. Complementarity may be “partial,” in which only some of the nucleic acids' bases are matched according to the base pairing rules, or there may be “complete” or “total” complementarity between the nucleic acids. The degree of complementarity between nucleic acid strands can have significant effects on the efficiency and strength of hybridization between nucleic acid strands under defined conditions. This is of particular importance for methods that depend upon binding between nucleic acids.

As used herein, the term “hybridization” is used in reference to the pairing of complementary nucleic acids. Hybridization and the strength of hybridization (i.e., the strength of the association between the nucleic acids) is influenced by such factors as the degree of complementary between the nucleic acids, stringency of the conditions involved, and the Tm of the formed hybrid. “Hybridization” methods involve the annealing of one nucleic acid to another, complementary nucleic acid, i.e., a nucleic acid having a complementary nucleotide sequence.

Hybridization is carried out in conditions permitting specific hybridization. The length of the complementary sequences and GC content affects the thermal melting point Tm of the hybridization conditions necessary for obtaining specific hybridization of the target site to the target nucleic acid. Hybridization may be carried out under stringent conditions. The phrase “stringent hybridization conditions” refers to conditions under which a probe will hybridize to its target subsequence, typically in a complex mixture of nucleic acid, but to no other sequences at a detectable or significant level. Stringent conditions are sequence-dependent and will be different in different circumstances. Stringent conditions are those in which the salt concentration is less than about 1.0 M sodium ion, such as less than about 0.01 M, including from about 0.001 M to about 1.0 M sodium ion concentration (or other salts) at a pH between about 6 to about 8 and the temperature is in the range of about 20° C. to about 65° C. Stringent conditions may also be achieved with the addition of destabilizing agents, such as but not limited to formamide.

The formation of a duplex molecule with all perfectly formed hydrogen-bonds between corresponding nucleotides is referred as “matched” or “perfectly matched”, and duplexes with single or several pairs of nucleotides that do not correspond are referred to as “mismatched.” Any combination of single-stranded RNA or DNA molecules can form duplex molecules (DNA:DNA, DNA:RNA, RNA:DNA, or RNA:RNA) under appropriate experimental conditions.

The phrase “selectively (or specifically) hybridizing” refers to the binding, duplexing, or hybridizing of a molecule only to a particular nucleotide sequence under stringent hybridization conditions when that sequence is present in a complex mixture (e.g. total cellular or library DNA or RNA).

Those of ordinary skill in the art will readily recognize that alternative hybridization and wash conditions can be utilized to provide conditions of similar stringency and will recognize that the combination of parameters is much more important than the measure of any single parameter.

A “substitution” results from the replacement of one or more nucleotides by different nucleotides, as compared to an example nucleotide sequence e.g., a WT nucleotide sequence.

A “deletion” is defined as a change in nucleotide sequence in which one or more nucleotide residues are absent as compared to an example nucleotide sequence, e.g., a WT nucleotide sequence. In the context of a polynucleotide sequence, a deletion can involve deletion of 2, 5, 10, up to 20, up to 30 or up to 50 or more nucleotides from the polynucleotide sequence being modified.

An “insertion” or “addition” is that change in a nucleotide sequence which has resulted in the addition of one or more nucleotide residues as compared to an example nucleotide sequence. In the context of a polynucleotide sequence, an insertion or addition may be of up to 10, up to 20, up to 30 or up to 50 or more nucleotides.

The terms “drop,” “droplet,” and “microdroplet” are used interchangeably herein, to refer to small, generally spherically structures, containing at least a first fluid phase, e.g., an aqueous phase (e.g., water), bounded by a second fluid phase (e.g., oil) which is immiscible with the first fluid phase. In some embodiments, droplets according to the present disclosure may contain a first fluid phase, e.g., oil, bounded by a second immiscible fluid phase, e.g. an aqueous phase fluid (e.g., water). In some embodiments, the second fluid phase will be an immiscible phase carrier fluid. Thus droplets according to the present disclosure may be provided as aqueous-in-oil emulsions or oil-in-aqueous emulsions. Droplets according to the present disclosure may be formed as multiple emulsions, such as double or higher level emulsions. In some embodiments, the subject droplets have a dimension, e.g., a diameter, of or about 1.0 μm to 1000 μm, inclusive, such as 1.0 μm to 750 μm, 1.0 μm to 500 μm, 1.0 μm to 100 μm, 1.0 μm to 10 μm, or 1.0 μm to 5 μm, inclusive. In some embodiments, discrete entities as described herein have a dimension, e.g., diameter, of or about 1.0 μm to 5 μm, 5 μm to 10 μm, 10 μm to 50 μm, 50 μm to 100 μm, 100 μm to 500 μm, 500 μm to 750 μm, or 750 μm to 1000 μm, inclusive. Furthermore, in some embodiments, discrete entities as described herein have a volume ranging from about 1 fL to 1 nL, inclusive, such as from 1 fL to 100 pL, 1 fL to 10 pL, 1 fL to 1 pL, 1 fL to 100 fL, or 1 fL to 10 fL, inclusive. In some embodiments, discrete entities as described herein have a volume of 1 fL to 10 fL, 10 fL to 100 fL, 100 fL to 1 pL, 1 pL to 10 pL, 10 pL to 100 pL or 100 pL to 1 nL, inclusive. Droplets according to the present disclosure generally have an internal volume ranging from about 0.001 picoliters to about 10,000 picoliters in volume, e.g., from about 1 picoliter to about 1000 picoliters, or from about 1 picoliter to about 100 picoliters. For example, in some embodiments, droplets according to the present disclosure have a volume ranging from about 0.001 picoliter to about 0.01 picoliter, from about 0.01 picoliter to about 0.1 picoliter, from about 0.1 picoliter to about 1 picoliter, from about 1 picoliter to about 10 picoliters, from about 10 picoliters to about 100 picoliters, from about 100 picoliters to about 1000 picoliters, or from about 1000 picoliters to about 10,000 picoliters. Droplets according to the present disclosure may be used to encapsulate cells, nucleic acids (e.g., DNA), enzymes, reagents, and a variety of other components. The term droplet may be used to refer to a droplet produced in, on, or by a microfluidic device and/or flowed from or applied by a microfluidic device.

As used herein, the term “carrier fluid” refers to a fluid configured or selected to contain one or more droplets as described herein. A carrier fluid may include one or more substances and may have one or more properties, e.g., viscosity, which allow it to be flowed through a microfluidic device or a portion thereof, such as a delivery orifice. In some embodiments, carrier fluids include, for example: oil or water, and may be in a liquid or gas phase. Suitable carrier fluids are described in greater detail herein.

As used in the claims, the term “comprising”, which is synonymous with “including”, “containing”, and “characterized by”, is inclusive or open-ended and does not exclude additional, unrecited elements and/or method steps. “Comprising” is a term of art that means that the named elements and/or steps are present, but that other elements and/or steps can be added and still fall within the scope of the relevant subject matter.

As used herein, the phrase “consisting of” excludes any element, step, and/or ingredient not specifically recited. For example, when the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of the related disclosure or claim to the specified materials and/or steps, plus those that do not materially affect the basic and novel characteristic(s) of the disclosed and/or claimed subject matter.

With respect to the terms “comprising”, “consisting essentially of”, and “consisting of”, where one of these three terms is used herein, the presently disclosed subject matter can include the use of either of the other two terms.

An example of a need in the art addressed by the present disclosure, is the need for uniform amplification of nucleic acid template molecules. Currently available MDA methods can lead to substantial amplification bias. As a result, sequencing results obtained therefrom are often inaccurate and unreliable. For example, amplified genomic data prepared according to conventional methods may not provide an accurate representation of the DNA present in the genome of a particular cell. According to aspects of the methods and compositions described herein, genomic DNA may be amplified in a significantly more uniform manner such that a more accurate representation of the genomic nucleic acids present in a biological sample, e.g., a cell, may be determined.

The present disclosure is directed in part to digital droplet multiple displacement amplification (ddMDA). “ddMDA” generally refers to compartmentalizing the amplification reaction of nucleic acid template molecule(s) in a single droplet reaction compartment (e.g., microdroplet), which results in generally parallel or uniform amplification of the nucleic acid template molecules. In some embodiments, the amplification reaction refers to amplifying a single (or a very few, e.g., 10 or less, such as 5 or less) nucleic acid template molecule in a single microdroplet. In other embodiments, the amplification reaction may amplify multiple nucleic acid template molecules in a single nucleic acid template molecule. Since the nucleic acid template molecules are physically isolated from one another, the molecules are able to amplify to saturation without competing with other molecules for resources. This yields a generally uniform representation of all genomic sequences. In some embodiments, each single nucleic acid template molecule is physically isolated from other nucleic acid template molecules such that amplification of the nucleic acid template molecule occurs irrespective of what is occurring outside of the microdroplet. Furthermore, confining a single nucleic acid template molecule in a single microdroplet negates the need to share similar resources (e.g., primers, reagents, polymerase enzymes).

In some embodiments, the ddMDA reaction amplifies nucleic acid template molecules compartmentalized in reaction chambers (e.g., microdroplets) having picoliter interior volumes. In some examples, compartmentalizing reactions of the nucleic acid template molecules may be achieved by emulsifying the solution containing the nucleic acid template molecules to be amplified with oil with vigorous shaking. If a suitable surfactant is present, stable aqueous droplets suspended in oil are produced, each of which amplifies a single nucleic acid template molecule. Alternatively, in other examples, compartmentalizing reactions in microdroplets can be achieved by using microfluidic emulsification techniques.

As described herein, amplification “bias” refers generally to unequal or disproportionate amplification of select genomic sequences. Such amplification bias generally reduces the quality and quantity of next-generation sequencing data by rendering an inaccurate representation of the genomic sequence.

As described herein, the term “nucleic acid template molecule” generally refers to a nucleic acid molecule which is used as a template for an MDA reaction as described herein. In some examples, the nucleic acid may refer to deoxyribose nucleic acid (DNA), ribonucleic acid (RNA), or complementary DNA (cDNA). In some embodiments, a cDNA molecule may be generated from an RNA molecule and may subsequently serve as a nucleic acid template molecule for an MDA reaction as described herein. This technique may be used, for example, to sequence the genome of one or more RNA viruses.

According to one embodiment, the method may non-specifically amplify a nucleic acid template molecule. As used in this context, the term “non-specifically” refers generally to amplifying a nucleic acid template molecule without bias or preference to a specific DNA sequence. As a result, non-specific amplification yields generally uniform amplification, e.g., of a genome of a cell.

To determine whether an MDA amplification product is present in a particular droplet, the MDA amplification products may be detected through an assay probing the liquid of the drop, such as by staining the solution with an intercalating dye, like SybrGreen or ethidium bromide, hybridizing the MDA amplification products to a solid substrate, such as a bead (e.g., magnetic or fluorescent beads, such as Luminex beads), or detecting them through an intermolecular reaction, such as FRET. These dyes, beads, and the like are each examples of a “detection component,” a term that is used broadly and generically herein to refer to any component that is used to detect the presence or absence of MDA amplification product(s).

In some examples, the methods and compositions described herein may be used to study uncultivable microbes through unbiased amplification and sequencing of genomic DNA. In other examples, the methods and compositions described herein may be used to analyze CNV by amplifying and sequencing the genomic DNA of individual cancer cells. In other examples, the methods and compositions described herein may be used to amplify forensic DNA for analysis. In other examples, the methods and compositions described herein may be used to amplify DNA from precious samples (e.g., ancient DNA) for sequencing. Forensic investigation, for instance, requires amplification and sequencing of samples well below the sensitivity limits of routine DNA analysis. Incorporating ddMDA into these analysis methods may yield enhanced uniformity of whole genome amplification; thus, improving draft genomes and follow-on analyses of the data. In other examples, the methods and compositions described herein may be valuable for amplifying limited DNA samples. In addition, ddMDA on individual tumor cells may provide more accurate sequences of cancer-associated mutations or copy-number variant data, for tracking the progression and evolution of the disease.

Lysis

In order to obtain genomic DNA from one or more cells for amplification according to the methods provided herein, one or more lysing agents may be utilized, under conditions in which the cell(s) may be caused to burst, thereby releasing their genomes. Any convenient lysing agent may be employed, such as a suitable protease, e.g., proteinase K, or cytotoxins. In particular embodiments, cells may be incubated with lysis buffer containing detergents such as Triton X100 and/or proteinase K. The specific conditions in which the cell(s) may be caused to burst will vary depending on the specific lysing agent used. For example, if a protease, e.g., proteinase K, is incorporated as a lysing agent, the cell(s) may be heated to about 37-60° C. for at least about 20 min to lyse the cells and to allow the proteinase K to digest cellular proteins, after which they may be heated to about 95° C. for about 5-10 min to deactivate the protease, e.g., proteinase K.

In certain aspects, cell lysis may also, or instead, rely on techniques that do not involve addition of lysing agent. For example, lysis may be achieved by mechanical techniques that may employ various geometric features to effect piercing, shearing, abrading, etc. of cells. Other types of mechanical breakage such as acoustic techniques may also be used. Further, thermal energy can also be used to lyse cells. Any convenient means of effecting cell lysis may be employed in the methods described herein.

In order to effectively amplify nucleic acids from target components, a microfluidics system may include a cell lysing or viral protein coat-disrupting module to free nucleic acids prior to providing the sample to an amplification module. Cell lysing modules may rely on chemical, thermal, and/or mechanical means to effect cell lysis. Because the cell membrane consists of a lipid double-layer, lysis buffers containing surfactants can solubilize the lipid membranes. Typically, the lysis buffer will be introduced directly to a lysis chamber via an external port so that the cells are not prematurely lysed during sorting or other upstream process.

In cases where organelle integrity is necessary, chemical lysis methods may be inappropriate. Mechanical breakdown of the cell membrane by shear and wear is appropriate in certain applications. Lysis modules relying mechanical techniques may employ various geometric features to effect piercing, shearing, abrading, etc. of cells entering the module. Other types of mechanical breakage such as acoustic techniques may also yield appropriate lysate. Further, thermal energy can also be used to lyse cells such as bacteria, yeasts, and spores. Heating disrupts the cell membrane and the intracellular materials are released. In order to enable subcellular fractionation in microfluidic systems a lysis module may also employ an electrokinetic technique or electroporation. Electroporation creates transient or permanent holes in the cell membranes by application of an external electric field that induces changes in the plasma membrane and disrupts the transmembrane potential. In microfluidic electroporation devices, the membrane may be permanently disrupted, and holes on the cell membranes sustained to release desired intracellular materials released.

Fragmentation

Fragmentation may be performed in order to separate or isolate a nucleic acid molecule or molecules into separate fragments. Fragmentation of a nucleic acid molecule may be achieved by thermal heating, electromagnetic irradiation, sonication, acoustic shearing, restriction digestion, needle shearing, point-sink shearing, or using French pressure.

In some embodiments, the cells of the biological sample may be fragmented, prior to the encapsulating step, in order to isolate the nucleic acid template molecule.

In certain aspects, the amount of nucleic acid template molecule provided in a droplet prior to amplifying via MDA to produce MDA products may be as little as 100 femtograms, e.g., 50 femtograms or less, 10 femtograms or less, or 5 femtograms or less. In some embodiments, the amount of nucleic acid template molecule provided in a droplet prior to amplifying via MDA to produce MDA products may be from about 1 femtogram to about 5 femtograms, from about 5 femtograms to about 10 femtograms, from about 10 femtograms to about 50 femtograms, or more.

Multiple Displacement Amplification

As summarized above, in practicing methods of the invention MDA may be used to amplify nucleic acids, e.g., genomic DNA, in a generally unbiased and non-specific manner for downstream analysis, e.g., via next generation sequencing.

An exemplary embodiment of a method according to the present disclosure includes encapsulating in a microdroplet a nucleic acid template molecule obtained from a biological sample, introducing MDA reagents and a plurality of MDA primers into the microdroplet, and incubating the microdroplet under conditions effective for the production of MDA amplification products, wherein the incubating is effective to produce MDA amplification products from the nucleic acid template molecule. In some embodiments the encapsulating and introducing steps occur as a single step, e.g., where the nucleic acid template molecule is mixed with MDA reagents and a plurality of MDA primers and emulsified, e.g., using a flow focusing element of a microfluidic device.

The conditions of MDA-based assays described herein may vary in one or more ways. For instance, the number of MDA primers that may be added to (or encapsulated in) a microdroplet may vary. The term “primer” refers to one or more primer and refers to an oligonucleotide, whether occurring naturally, as in a purified restriction digest, or produced synthetically, which is capable of acting as a point of initiation of synthesis along a complementary strand when placed under conditions in which synthesis of a primer extension product which is complementary to a nucleic acid strand is catalyzed. Such conditions include the presence of four different deoxyribonucleoside triphosphates and a polymerization-inducing agent such as a suitable DNA polymerase (e.g., (D29 Polymerase or Bst Polymerase), in a suitable buffer (“buffer” includes substituents which are cofactors, or which affect pH, ionic strength, etc.), and at a suitable temperature. The primer is preferably single-stranded for maximum efficiency in amplification. In the context of MDA, random hexamer primers are regularly utilized.

The complement of a nucleic acid sequence as used herein refers to an oligonucleotide which, when aligned with the nucleic acid sequence such that the 5′ end of one sequence is paired with the 3′ end of the other, is in “antiparallel association.” Complementarity need not be perfect; stable duplexes may contain mismatched base pairs or unmatched bases. Those skilled in the art of nucleic acid technology can determine duplex stability empirically considering a number of variables including, for example, the length of the oligonucleotide, percent concentration of cytosine and guanine bases in the oligonucleotide, ionic strength, and incidence of mismatched base pairs.

The number of MDA primers that may be added to (or encapsulated in) a microdroplet may range from about 1 to about 500 or more, e.g., about 2 to 100 primers, about 2 to 10 primers, about 10 to 20 primers, about 20 to 30 primers, about 30 to 40 primers, about 40 to 50 primers, about 50 to 60 primers, about 60 to 70 primers, about 70 to 80 primers, about 80 to 90 primers, about 90 to 100 primers, about 100 to 150 primers, about 150 to 200 primers, about 200 to 250 primers, about 250 to 300 primers, about 300 to 350 primers, about 350 to 400 primers, about 400 to 450 primers, about 450 to 500 primers, or about 500 primers or more.

Such primers and/or reagents may be added to a microdroplet in one step, or in more than one step. For instance, the primers may be added in two or more steps, three or more steps, four or more steps, or five or more steps. Where a lysing agent is utilized, regardless of whether the primers are added in one step or in more than one step, they may be added after the addition of a lysing agent, prior to the addition of a lysing agent, or concomitantly with the addition of a lysing agent. When added before or after the addition of a lysing agent, the MDA primers may be added in a separate step from the addition of a lysing agent.

Once primers have been added to a microdroplet, the microdroplet may be incubated under conditions sufficient for MDA. The microdroplet may be incubated on the same microfluidic device as was used to add the primer(s), or may be incubated on a separate device. In certain embodiments, incubating the microdroplet under conditions sufficient for MDA amplification is performed on the same microfluidic device used for cell lysis. Incubating the microdroplets may take a variety of forms, for example microdroplets may be incubated at a constant temperature, e.g., 30 deg. C., e.g., for about 8 to about 16 hours.

Although the methods described herein for producing MDA amplification products do not require the use of specific probes, the methods of the invention may also include introducing one or more probes to the microdroplet. As used herein with respect to nucleic acids, the term “probe” generally refers to a labeled oligonucleotide which forms a duplex structure with a sequence in the target nucleic acid, due to complementarity of at least one sequence in the probe with a sequence in the target region. The probe, preferably, does not contain a sequence complementary to sequence(s) used to prime the MDA reaction. The number of probes that are added may be from about one to 500, e.g., about 1 to 10 probes, about 10 to 20 probes, about 20 to 30 probes, about 30 to 40 probes, about 40 to 50 probes, about 50 to 60 probes, about 60 to 70 probes, about 70 to 80 probes, about 80 to 90 probes, about 90 to 100 probes, about 100 to 150 probes, about 150 to 200 probes, about 200 to 250 probes, about 250 to 300 probes, about 300 to 350 probes, about 350 to 400 probes, about 400 to 450 probes, about 450 to 500 probes, or about 500 probes or more. The probe(s) may be introduced into the microdroplet prior to, subsequent with, or after the addition of the one or more primer(s).

In certain embodiments, an MDA based assay may be used to detect the presence of certain RNA transcripts present in cells or to sequence the genome of one or more RNA viruses. In such embodiments, MDA reagents may be added to the microdroplet using any of the methods described herein. Prior to or after addition (or encapsulation) of the MDA reagents, the microdroplet may be incubated under conditions allowing for reverse transcription followed by conditions allowing for MDA as described herein. The microdroplet may be incubated on the same microfluidic device as is used to add the MDA reagents, or may be incubated on a separate device. In certain embodiments, incubating the microdroplet under conditions allowing for MDA is performed on the same microfluidic device used to encapsulate and/or lyse one or more cells.

In certain embodiments, the reagents added to the microdroplet for MDA further includes a fluorescent DNA probe capable of detecting MDA amplification products. Any suitable fluorescent DNA probe can be used including, but not limited to SYBR Green, TaqMan®, Molecular Beacons and Scorpion probes. In certain embodiments, the reagents added to the microdroplet include more than one DNA probe, e.g., two fluorescent DNA probes, three fluorescent DNA probes, or four fluorescent DNA probes. The use of multiple fluorescent DNA probes allows for the concurrent measurement of MDA amplification products in a single reaction.

Types of Microdroplets

In practicing the methods of the present invention, the composition and nature of the microdroplets may vary. For instance, in certain aspects, a surfactant may be used to stabilize the microdroplets. Accordingly, a microdroplet may involve a surfactant stabilized emulsion. Any convenient surfactant that allows for the desired reactions to be performed in the drops may be used. In other aspects, a microdroplet is not stabilized by surfactants or particles.

The surfactant used depends on a number of factors such as the oil and aqueous phases (or other suitable immiscible phases, e.g., any suitable hydrophobic and hydrophilic phases) used for the emulsions. For example, when using aqueous droplets in a fluorocarbon oil, the surfactant may have a hydrophilic block (PEG-PPO) and a hydrophobic fluorinated block (Krytox FSH). If, however, the oil was switched to be a hydrocarbon oil, for example, the surfactant would instead be chosen so that it had a hydrophobic hydrocarbon block, like the surfactant ABIL EM90. In selecting a surfactant, desirable properties that may be considered in choosing the surfactant may include one or more of the following: (1) the surfactant has low viscosity; (2) the surfactant is immiscible with the polymer used to construct the device, and thus it doesn't swell the device; (3) biocompatibility; (4) the assay reagents are not soluble in the surfactant; (5) the surfactant exhibits favorable gas solubility, in that it allows gases to come in and out; (6) the surfactant has a boiling point higher than the temperature used for MDA or that of any other reactions the droplets will be exposed to; (7) the emulsion stability; (8) that the surfactant stabilizes drops of the desired size; (9) that the surfactant is soluble in the carrier phase and not in the droplet phase; (10) that the surfactant has limited fluorescence properties; and (11) that the surfactant remains soluble in the carrier phase over a range of temperatures.

Other surfactants can also be envisioned, including ionic surfactants. Other additives can also be included in the oil to stabilize the drops, including polymers that increase droplet stability at temperatures above 35° C.

In some embodiments a suitable surfactant is a PEG-PFPE amphiphilic block copolymer surfactant. Such a surfactant may be utilized in a shaken emulsion MDA method. In some embodiments a suitable oil for use in the preparation of microdroplets, e.g., shaken emulsion microdroplets is the fluorinated oil HFE-7500.

In some embodiments, the nucleic acid template molecule may be encapsulated in a multiple-emulsion microdroplet, wherein each multiple-emulsion microdroplet includes a first miscible phase fluid surrounded by an immiscible shell, wherein the multiple-emulsion microdroplet is positioned in a second miscible phase carrier fluid. In some embodiments, the sample may be diluted prior to encapsulation, e.g., so as to encapsulate a controlled number of cells, viruses, and/or nucleic acids in the multiple-emulsion microdroplets. Nucleic acid amplification reagents, e.g., MDA reagents, may be added to the multiple-emulsion microdroplets at the time of encapsulation or added to the multiple-emulsion microdroplets at a later time using one or more of the methods described herein. The multiple-emulsion microdroplets are then subjected to nucleic acid amplification conditions. In some embodiments, a label is added such that if a multiple-emulsion microdroplet contains a nucleic acid template molecule, the multiple-emulsion microdroplet becomes detectably labeled, e.g., fluorescently labeled as a result of a fluorogenic assay, such as Sybr staining of amplified DNA. To recover the amplified nucleic acids, the detectably labeled multiple-emulsion microdroplets may be sorted using microfluidic (e.g., dielectrophoresis, membrane valves, etc.) or non-microfluidic techniques (e.g., FACS).

In some embodiments, the microdroplet includes a nucleic acid template molecule encapsulated or compartmentalized within the microdroplet and an MDA mixture including a DNA polymerase enzyme, a plurality of MDA reagents, and a plurality of MDA primers. In other aspects, the microdroplet may further include a detection component.

In some embodiments, the microdroplet includes a single nucleic acid template molecule. In other embodiments, there may be multiple nucleic acid template molecules compartmentalized in a single microdroplet.

In some embodiments, the microdroplet, prior to the introducing and incubating steps, does not include more than one nucleic acid template molecule. In other embodiments, the microdroplet, prior to introducing and incubating steps, may include multiple nucleic acid template molecules.

In some embodiments, the number of nucleic acid template molecules to be amplified can be varied by controlling the number of microdroplets which are generated. In other embodiments, the size of the microdroplet may be varied in order to obtain a predetermined amount of MDA amplification products derived from the nucleic acid template molecule.

In some embodiments, both microfluidic and non-microfluidic methods may be utilized to generate microdroplets to provide MDA amplification products.

In some embodiments, the starting amount of the nucleic acid template molecule (prior to amplification) is low, e.g., not more than 10 fg (e.g., not more than 5 fg or not more than 1 fg) of the nucleic acid template molecule is encapsulated in the microdroplet. In some embodiments, between about 10 fg and about 1 fg (e.g., between about 5 fg and 1 fg) is encapsulated in the microdroplet prior to amplification. In some embodiments, the microdroplet may also include a detection component, such as a fluorescent reporter. The fluorescent reporter may indicate when a specific microdroplet undergoes amplification. In contrast to ddPCR, ddMDA does not rely upon specific primers and probes to amplify only specific nucleic acid template molecule(s). In some embodiments, ddMDA yields approximately one fluorescent droplet for every genomic fragment amplifiable within the MDA reaction. As ddMDA does not require specific probes, the ddMDA method enables the quantification of both known and unknown genomic sequences. These advantages make ddMDA valuable for quantitating DNA in low-abundance settings, such as clean rooms and extra-terrestrial habitats. When used with ddPCR, ddMDA is also effective for detecting fragmentation and contamination during DNA amplification.

In some embodiments, the microdroplet may include amplicons produced from the encapsulated nucleic acid template molecule. As described herein, “amplicons” generally refers to an amplification product of products, which are the product of natural or artificial amplification. The term amplicon may refer generally to one or more copies of a genomic sequence, such as an RNA or DNA sequence.

In some embodiments, the internal volume of the microdroplet may be about 0.01 pL or less, about 0.1 pL or less, 1 pL or less, about 5 pL or less, 10 pL or less, 100 pL or less, or 1000 pL or less. In some embodiments, the internal volume of the microdroplet may be about 1 fL or less, about 10 fL or less, or 100 fL or less. In some embodiments, the internal volume of the microdroplet may encompass a liquid volume which ranges between picoliters and femotliters (e.g., about 0.001 pL to about 1000 pL). In some embodiments, the internal volume of the microdroplet extends strictly below the nanoliter level (e.g., strictly picoliter, strictly femtoliter, or combination thereof).

In some embodiments, the initial concentration of the nucleic acid template molecule(s) in the microdroplet is from about 0.001 pg to about 10 pg, e.g., from about 0.01 pg to about 1 pg, or from about 0.1 pg to about 1 pg.

In some examples, the microdroplets may be created as polydisperse microdroplets or monodisperse microdroplets.

Adding Reagents to Microdroplets

In practicing the subject methods, a number of reagents may need to be added to the microdroplets, in one or more steps (e.g., about 2, about 3, about 4, or about 5 or more steps). The means of adding reagents to the microdroplets may vary in a number of ways. Approaches of interest include, but are not limited to, those described by Ahn, et al., Appl. Phys. Lett. 88, 264105 (2006); Priest, et al., Appl. Phys. Lett. 89, 134101 (2006); Abate, et al., PNAS, Nov. 9, 2010 vol. 107 no. 45 19163-19166; and Song, et al., Anal. Chem., 2006, 78 (14), pp 4839-4849; the disclosures of which are incorporated herein by reference.

For instance, a reagent may be added to a microdroplet by a method involving merging a microdroplet with a second microdroplet that contains the reagent(s). The reagent(s) that are contained in the second microdroplet may be added by any convenient means, specifically including those described herein. This droplet may be merged with the first microdroplet to create a microdroplet that includes the contents of both the first microdroplet and the second microdroplet.

One or more reagents may also, or instead, be added using techniques such as droplet coalescence, or picoinjection. In droplet coalescence, a target drop (i.e., the microdroplet) may be flowed alongside a microdroplet containing the reagent(s) to be added to the microdroplet. The two microdroplets may be flowed such that they are in contact with each other, but not touching other microdroplets. These drops may then be passed through electrodes or other means of applying an electrical field, wherein the electric field may destabilize the microdroplets such that they are merged together.

Reagents may also, or instead, be added using picoinjection. In this approach, a target drop (i.e., the microdroplet) may be flowed past a channel containing the reagent(s) to be added, wherein the reagent(s) are at an elevated pressure. Due to the presence of the surfactants, however, in the absence of an electric field, the microdroplet will flow past without being injected, because surfactants coating the microdroplet may prevent the fluid(s) from entering. However, if an electric field is applied to the microdroplet as it passes the injector, fluid containing the reagent(s) will be injected into the microdroplet. The amount of reagent added to the microdroplet may be controlled by several different parameters, such as by adjusting the injection pressure and the velocity of the flowing drops, by switching the electric field on and off, and the like.

In other aspects, one or more reagents may also, or instead, be added to a microdroplet by a method that does not rely on merging two droplets together or on injecting liquid into a drop. Rather, one or more reagents may be added to a microdroplet by a method involving the steps of emulsifying a reagent into a stream of very small drops, and merging these small drops with a target microdroplet. Such methods shall be referred to herein as “reagent addition through multiple-drop coalescence.” These methods take advantage of the fact that due to the small size of the drops to be added compared to that of the target drops, the small drops will flow faster than the target drops and collect behind them. The collection can then be merged by, for example, applying an electric field. This approach can also, or instead, be used to add multiple reagents to a microdroplet by using several co-flowing streams of small drops of different fluids. To enable effective merger of the tiny and target drops, it is important to make the tiny drops smaller than the channel containing the target drops, and also to make the distance between the channel injecting the target drops from the electrodes applying the electric field sufficiently long so as to give the tiny drops time to “catch up” to the target drops. If this channel is too short, not all tiny drops will merge with the target drop and adding less reagent than desired. To a certain degree, this can be compensated for by increasing the magnitude of the electric field, which tends to allow drops that are farther apart to merge. In addition to making the tiny drops on the same microfluidic device, they can also, or instead, be made offline using another microfluidic drop maker or through homogenization and then injecting them into the device containing the target drops.

Accordingly, in certain aspects a reagent is added to a microdroplet by a method involving emulsifying the reagent into a stream of droplets, wherein the droplets are smaller than the size of the microdroplet; flowing the droplets together with the microdroplet; and merging a droplet with the microdroplet. The diameter of the droplets contained in the stream of droplets may vary ranging from about 75% or less than that of the diameter of the microdroplet, e.g., the diameter of the flowing droplets is about 75% or less than that of the diameter of the microdroplet, about 50% or less than that of the diameter of the microdroplet, about 25% or less than that of the diameter of the microdroplet, about 15% or less than that of the diameter of the microdroplet, about 10% or less than that of the diameter of the microdroplet, about 5% or less than that of the diameter of the microdroplet, or about 2% or less than that of the diameter of the microdroplet. In certain aspects, a plurality of flowing droplets may be merged with the microdroplet, such as 2 or more droplets, 3 or more, 4 or more, or 5 or more. Such merging may be achieved by any convenient means, including but not limited to by applying an electric field, wherein the electric field is effective to merge the flowing droplet with the microdroplet.

As a variation of the above-described methods, the fluids may be jetting. That is, rather than emulsifying the fluid to be added into flowing droplets, a long jet of this fluid can be formed and flowed alongside the target microdroplet. These two fluids can then be merged by, for example, applying an electric field. The result is a jet with bulges where the microdroplets are, which may naturally break apart into microdroplets of roughly the size of the target microdroplets before the merger, due to the Rayleigh plateau instability. A number of variants are contemplated. For instance, one or more agents may be added to the jetting fluid to make it easier to jet, such as gelling agents and/or surfactants. Moreover, the viscosity of the continuous fluid could also be adjusted to enable jetting, such as that described by Utada, et al., Phys. Rev. Lett. 99, 094502 (2007), the disclosure of which is incorporated herein by reference.

In other aspects, one or more reagents may be added using a method that uses the injection fluid itself as an electrode, by exploiting dissolved electrolytes in solution.

In another aspect, a reagent is added to a drop (e.g., a microdroplet) formed at an earlier time by enveloping the drop to which the reagent is be added (i.e., the “target drop”) inside a drop containing the reagent to be added (the “target reagent”). In certain embodiments such a method is carried out by first encapsulating the target drop in a shell of a suitable hydrophobic phase, e.g., oil, to form a double emulsion. The double emulsion is then encapsulated by a drop containing the target reagent to form a triple emulsion. To combine the target drop with the drop containing the target reagent, the double emulsion is then burst open using any suitable method, including, but not limited to, applying an electric field, adding chemicals that destabilizes the droplet interface, flowing the triple emulsion through constrictions and other microfluidic geometries, applying mechanical agitation or ultrasound, increasing or reducing temperature, or by encapsulating magnetic particles in the drops that can rupture the double emulsion interface when pulled by a magnetic field. These and related methods are described in Published PCT application WO2014/028378, the disclosure of which is incorporated by reference herein in its entirety and for all purposes.

Detecting MDA Products

In practicing the subject methods, the manner in which MDA amplification products may be detected may vary. A variety of different detection components may be used in practicing the subject methods, including using fluorescent dyes known in the art. Fluorescent dyes may typically be divided into families, such as fluorescein and its derivatives; rhodamine and its derivatives; cyanine and its derivatives; coumarin and its derivatives; Cascade Blue and its derivatives; Lucifer Yellow and its derivatives; BODIPY and its derivatives; and the like. Exemplary fluorophores include indocarbocyanine (C3), indodicarbocyanine (C5), Cy3, Cy3.5, Cy5, Cy5.5, Cy7, Texas Red, Pacific Blue, Oregon Green 488, Alexa fluor-355, Alexa Fluor 488, Alexa Fluor 532, Alexa Fluor 546, Alexa Fluor-555, Alexa Fluor 568, Alexa Fluor 594, Alexa Fluor 647, Alexa Fluor 660, Alexa Fluor 680, JOE, Lissamine, Rhodamine Green, BODIPY, fluorescein isothiocyanate (FITC), carboxy-fluorescein (FAM), phycoerythrin, rhodamine, dichlororhodamine (dRhodamine), carboxy tetramethylrhodamine (TAMRA), carboxy-X-rhodamine (ROX), LIZ, VIC, NED, PET, SYBR, PicoGreen, RiboGreen, and the like. Descriptions of fluorophores and their use, can be found in, among other places, R. Haugland, Handbook of Fluorescent Probes and Research Products, 9th ed. (2002), Molecular Probes, Eugene, Oreg.; M. Schena, Microarray Analysis (2003), John Wiley & Sons, Hoboken, N.J.; Synthetic Medicinal Chemistry 2003/2004 Catalog, Berry and Associates, Ann Arbor, Mich.; G. Hermanson, Bioconjugate Techniques, Academic Press (1996); and Glen Research 2002 Catalog, Sterling, Va.

Copy Number Variation

Copy number variation (CNV) is a form of structural variation in the genome. As described herein, CNV generally refers to a variation in the DNA segments of the genome larger than 1 kbp. In some examples, the alteration of the genome may be either abnormal or, for certain genes, may be a normal variation in the number of copies of the one or more sections of the DNA. CNV corresponds to large regions of the genome that have been deleted (i.e., fewer than normal number) and large regions of the genome that have been duplicated (i.e., more than the normal number). CNVs may vary; however, shorter CNVs are generally more difficult to detect than longer CNVs.

CNV Analysis generally refers to analyzing the amplified MDA products to estimate the CNV (i.e., detect for variations in the number of copies of a particular DNA sequence).

Next-generation sequencing (NGS) may be utilized in connection with the MDA methods described herein to analyze CNV. Specific NGS techniques for detecting CNV may include CNV-seq, FREEC, readDepth, CNVnator, SegSeq, event-wise testing (EWT), rSW-seq, CNAnorm, cND, CNAseg, CNVer, CopySeq, JointSLM, and cn.MOPS. Other examples of NGS for identifying CNV in a genome includes pair-end mapping (PEM) based and depth of coverage (DOC) based methods. In some examples, PEM based methods may be suitable to detect CNVs of a smaller size.

In some embodiments, CNV may be performed on a small DNA segment, e.g., a single gene. In other embodiments, CNV may be performed on multiple genes.

In some examples, CNVs are associated with specific diseases, such as cancer. When a cancer patient is treated with one or more drugs, the cancerous cells are subjected to selective pressures which often cause the cancer cells to evolve to develop a resistance to the drug therapy. In order to evolve, cancer cells undergo forms of genetic mutation which can lead to drug therapy resistance, as demonstrated by copy-number variation (CNV), where regions of the cancer cell's genome can incorporate insertion (i.e., duplications) or deletions in the genome.

CNV analysis attempts to count the number of times that a particular sequence appears in the genome. The initial genetic material may be extracted from a cancer patient's cells and amplified in order to produce sufficient genetic material for performing a CNV analysis. By using the methods described herein, the genomic material may be uniformly amplified in order to provide accurate characterization of CNV in a population of nucleic acids, e.g., a population of nucleic acids derived from a single cell. By using the ddMDA methods described herein for producing MDA amplification products, minute quantities of DNA, including DNA derived from single cells, can be accurately and uniformly amplified.

In one example of CNV analysis, a population of nucleic acids is derived from a single cancer cell. The cancer cell is first isolated from the biological sample via sorting techniques such as either dilution or fluorescence-activated cell sorting (FACS) and placed into an individual reaction container (e.g., tube). Once isolated the cells are lysed and their genomes are fragmented into a size appropriate for ddMDA methods, such as 10⁴ bp-10⁶ bp fragments. The isolated and fragmented nucleic acid template molecules are then subject to ddMDA methods, which may include encapsulating the nucleic acid template molecule(s) within a microdroplet and adding MDA reagents, MDA primers, and a suitable DNA polymerase into the microdroplet. In some examples, the MDA mixture is emulsified so that a small number of amplification products are produced per microdroplet. Thereafter, the microdroplet is incubated to produce MDA amplification products from the nucleic acid template molecule(s). For example, the microdroplet is incubated at a temperature of 30° C. for 16 hours when performing bulk MDA or emulsion MDA.

In some embodiments, the size of the microdroplet may be varied in order to effect the predetermined amount of amplification per microdroplet as well as the total amount of amplified DNA for a downstream analysis. In other embodiments, the number of microdroplets may be varied as need to obtain the desired amount of amplification per molecule in order to effect the predetermined amount of amplification per microdroplet as well as the total amount of amplified DNA for a downstream analysis.

Thereafter, once the MDA amplification product is produced, the products may be analyzed to determine and/or quantitate CNV. In some embodiments, the DNA segments of the amplified products may be sequenced in order to quantify the number of times in which a particular sequence is repeated by aligning the DNA segment of the amplified products with human genome and comparing the different reads to assess insertions or deletions in the human genome. As this method for analyzing CNV in a population of nucleic acids utilizes uniformly amplified genomic material having minimal bias, any differences in the amplified products should be solely attributed to CNV, thereby allowing for an accurate CNV analysis.

While the methods described herein are described with respect to CNV analysis on DNA derived from cancer cells, the methods for CNV analysis may also be directed towards analyzing a number of other disease conditions, e.g., Alzheimer's disease, Parkinson disease, autism, Crohn disease, hemophilia, schizophrenia, and the like.

Next Generation Sequencing

As described herein, the term “next-generation sequencing” generally refers to advancements over standard DNA sequencing (e.g., Sanger sequencing). Although standard DNA sequencing enables the practitioner to determine the precise order of nucleotides in the DNA sequence, next-generation sequencing also provides parallel sequencing, during which millions of base pair fragments of DNA can be sequenced in unison. Standard DNA sequencing generally requires a single-stranded DNA template molecule, a DNA primer, and a DNA polymerase in order to amplify the DNA template molecule. Next-generation sequencing facilitates high-throughput sequencing, which allows for an entire genome to be sequenced in a significantly shorter period of time relative to standard DNA sequencing. Next-generation sequencing may also facilitate in identification of disease-causing mutations for diagnosis of pathological conditions. Next-generation sequencing may also provide information on the entire transcriptome of a sample in a single analysis without requiring prior knowledge of the genetic sequence.

In some examples, sequencing may involve mammalian cells, which include larger genomes than E. coli cells. E. coli genomes include ˜4.7 million base pairs. In contrast, the diploid human genome is complex and possesses over 6 billion base pairs. Due to its larger genome size, more fragments must be generated for fixed fragment length, which, in turn, will necessitate the generating of more droplets to ensure limiting Poisson encapsulation. For example, for a 10 kb fragment size, there will be 600,000 fragments, which will require ˜6 million droplets to ensure low loading rates for the ddMDA reaction. There is an immense amount of flexibility in the system, however, and this is well within the capabilities of ddMDA: Using ˜30 μm droplets, for example, a 6 million droplet emulsion will require ˜140 μL of ddMDA reagent and take about 30 min to generate with microfluidic flow focusing, both of which are reasonable. In addition, droplet volume, fragment length, and the method of emulsification can all be altered to optimize for the experiment. For example, higher-throughput droplet generation method such as parallel droplet generation (Romanowsky et al. (2012) “High throughput production of single core double emulsions in a parallelized microfluidic device.” Lab Chip, 12, 802), hierarchical droplet splitting (Abate, A. R. and Weitz, D. a (2011) “Faster multiple emulsification with drop splitting.” Lab Chip, 11, 1911-1915), and bubble triggered droplet generation (Abate, A. R. and Weitz, D. a (2011) “Air-bubble-triggered drop formation in microfluidics.” Lab Chip, 11, 1713-1716) each provide >10× throughput in droplet generation, and they can be used in combination.

Suitable Subjects

The subject methods may be applied to biological samples taken from a variety of different subjects. In many embodiments the subjects are “mammals” or “mammalian”, where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In many embodiments, the subjects are humans. The subject methods may be applied to human subjects of both genders and at any stage of development (i.e., neonates, infant, juvenile, adolescent, adult), where in certain embodiments the human subject is a juvenile, adolescent or adult. While the present invention may be applied to a human subject, it is to be understood that the subject methods may also be carried-out on other animal subjects (that is, in “non-human subjects”) such as, but not limited to, birds, mice, rats, dogs, cats, livestock and horses. Accordingly, it is to be understood that any subject in need of assessment according to the present disclosure is suitable.

Moreover, suitable subjects include those who have and those who have not been diagnosed with a condition, such as cancer. Suitable subjects include those that are and are not displaying clinical presentations of one or more cancers. In certain aspects, a subject may one that may be at risk of developing cancer, due to one or more factors such as family history, chemical and/or environmental exposure, genetic mutation(s) (e.g., BRCA1 and/or BRCA2 mutation), hormones, infectious agents, radiation exposure, lifestyle (e.g., diet and/or smoking), presence of one or more other disease conditions, and the like.

As described more fully above, a variety of different types of biological samples may be obtained from such subjects. In certain embodiments, whole blood is extracted from a subject. When desired, whole blood may be treated prior to practicing the subject methods, such as by centrifugation, fractionation, purification, and the like. The volume of the whole blood sample that is extracted from a subject may be 100 mL or less, e.g., about 100 mL or less, about 50 mL or less, about 30 mL or less, about 15 mL or less, about 10 mL or less, about 5 mL or less, or about 1 mL or less.

Devices

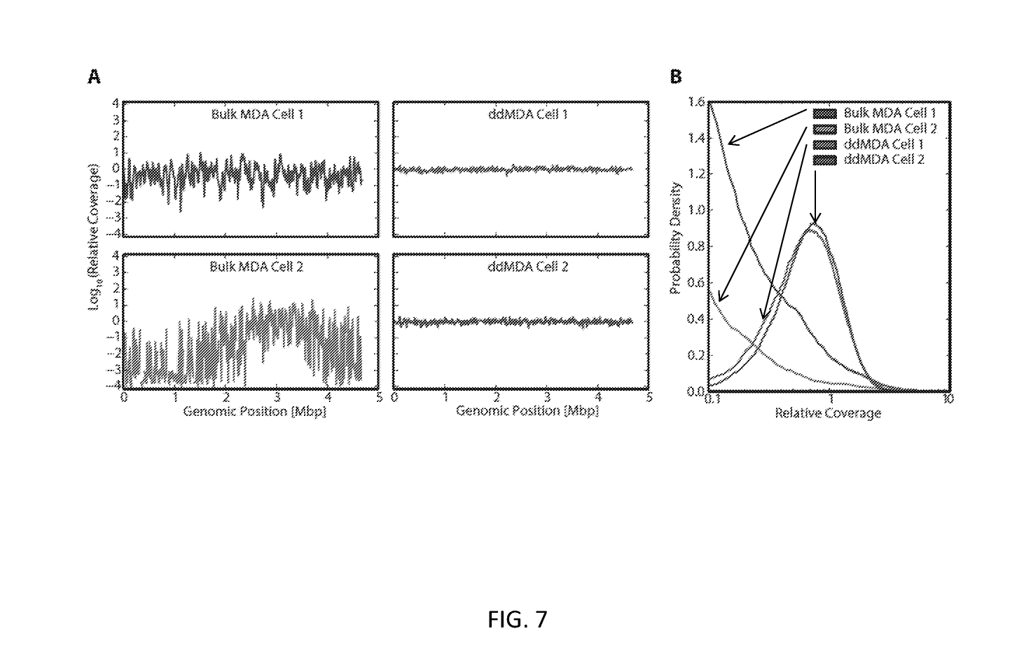

According to some embodiments, the methods described herein may be implemented using a microfluidic device. Microfluidic devices can contain a number of microchannels, valves, pumps, reactor, mixers and other components for producing the microdroplets. Suitable devices are described, for example, in PCT Application Publication WO2014/028378, the disclosure of which is incorporated by reference herein in its entirety and for all purposes. In addition, FIG. 7 illustrates an exemplary microfluidic device for implementing one or more aspects of the methods described herein. Specifically, FIG. 7 illustrates a microfluidic droplet maker which may be utilized to provide mono-disperse droplets for use in the disclosed methods.

As indicated above, embodiments of the invention employ microfluidics devices. Microfluidics devices of this invention may be implemented in various ways. In certain embodiments, for example, microfluidics devices have at least one “micro” channel. Such channels may have at least one cross-sectional dimension on the order of a millimeter or smaller (e.g., less than or equal to about 1 millimeter). For certain applications, this dimension may be adjusted; in some embodiments the at least one cross-sectional dimension is about 500 micrometers or less. In some embodiments, again as applications permit, the cross-sectional dimension is about 100 micrometers or less (or even about 10 micrometers or less—sometimes even about 1 micrometer or less). A cross-sectional dimension is one that is generally perpendicular to the direction of centerline flow, although it should be understood that when encountering flow through elbows or other features that tend to change flow direction, the cross-sectional dimension in play need not be strictly perpendicular to flow. It should also be understood that in some embodiments, a micro-channel may have two or more cross-sectional dimensions such as the height and width of a rectangular cross-section or the major and minor axes of an elliptical cross-section. Either of these dimensions may be compared against sizes presented here. Note that micro-channels employed in connection with the present disclosure may have two dimensions that are grossly disproportionate—e.g., a rectangular cross-section having a height of about 100-200 micrometers and a width on the order or a centimeter or more. Certain devices may employ channels in which the two or more axes are very similar or even identical in size (e.g., channels having a square or circular cross-section).

In view of the above, it should be understood that some of the principles and design features described herein can be scaled to larger devices and systems including devices and systems employing channels reaching the millimeter or even centimeter scale channel cross-sections. Thus, when describing some devices and systems as “microfluidic,” it is intended that the description apply equally, in certain embodiments, to some larger scale devices.

When referring to a microfluidic “device” it is generally intended to represent a single entity in which one or more channels, reservoirs, stations, etc. share a continuous substrate, which may or may not be monolithic. A microfluidics “system” may include one or more microfluidic devices and associated fluidic connections, electrical connections, control/logic features, etc. Aspects of microfluidic devices include the presence of one or more fluid flow paths, e.g., channels, having dimensions as discussed herein.

In certain embodiments, microfluidic devices of this invention provide a continuous flow of a fluid medium. Fluid flowing through a channel in a microfluidic device exhibits many interesting properties. Typically, the dimensionless Reynolds number is extremely low, resulting in flow that always remains laminar. Further, in this regime, two fluids joining will not easily mix, and diffusion alone may drive the mixing of two compounds.

### Exemplary Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-68 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below.
1. A method of non-specifically amplifying a nucleic acid template molecule, the method comprising:
   encapsulating in a microdroplet a nucleic acid template molecule obtained from a biological sample;
   introducing Multiple Displacement Amplification (MDA) reagents and a plurality of MDA primers into the microdroplet; and
   incubating the microdroplet under conditions effective for the production of MDA amplification products, wherein the incubating is effective to produce MDA amplification products from the nucleic acid template molecule.
2. The method of 1, wherein the microdroplet, prior to the introducing and incubating steps, does not include more than one nucleic acid template molecule.
3. The method of 1 or 2, wherein the MDA reagents comprise a Φ29 DNA polymerase or a Bst DNA polymerase.
4. The method of any one of 1-3, wherein the microdroplet has an internal volume of from about 0.001 picoliters to about 1000 picoliters.
5. The method of any one of 1-4, wherein the encapsulating comprises encapsulating in a plurality of microdroplets a plurality of nucleic acid template molecules obtained from one or more biological samples, the introducing comprises introducing MDA reagents and a plurality of MDA primers into each of the plurality of microdroplets, and the incubating comprises incubating the plurality of microdroplets under conditions effective for the production of MDA amplification products, wherein the incubating is effective to produce MDA amplification products from the nucleic acid template molecules.
6. The method of 5, wherein each of the plurality of microdroplets comprises zero or one, and not more than one, nucleic acid template molecule.
7. The method of any one of 1-6, wherein the nucleic acid template molecule, MDA reagents, and MDA primers are loaded into a droplet dispenser to form the microdroplet.
8. The method of any one of 1-7, wherein one or more steps are performed under microfluidic control.
9. The method of any one of 1-7, wherein the microdroplet is generated via shaken emulsion.
10. The method of any one of 1-7, wherein the microdroplet is generated via microfluidic emulsion.
11. The method of any one of 1-10, wherein one or more nucleic acids of the biological sample are fragmented to provide the nucleic acid template molecule.
12. The method of 11, wherein the fragmentation is via one of enzymatic fragmentation, heating, and sonication.
13. The method of any one of 1-10, wherein one or more cells of the biological sample are lysed to provide the nucleic acid template molecule.
14. The method of any one of 1-13, further comprising determining the sequence of the MDA amplification products via next-generating sequencing (NGS).
15. The method of any one of 1-14, wherein the MDA amplification products comprise a single MDA amplification product.
16. The method of any one of 1-14, wherein the MDA amplification products comprise a plurality of different MDA amplification products.
17. The method of any one of 1-12 and 14-16, wherein the biological sample comprises one or more cells.
18. The method of 17, wherein the one or more cells comprises one or more circulating tumor cells (CTC).
19. The method of any one of 1-18, further comprising a step of introducing a detection component into each microdroplet, wherein detection of the detection component indicates the presence of one more MDA amplification products.
20. The method of 19, wherein the detection component is detected based on a change in fluorescence.
21. The method of 5, wherein the internal volume of each microdroplet is of an approximately equal volume.
22. The method of 5, wherein the internal volume of each microdroplet is of a significantly different volume.
23. The method of 5, wherein the number of microdroplets corresponds to the number of nucleic acid template molecules.
24. The method of 5, wherein the number of nucleic acid template molecules to be amplified is varied by controlling the number of microdroplets generated.
25. The method of 5, wherein the size of each microdroplet is varied in order to obtain a predetermined amount of MDA amplification product derived from the nucleic acid template molecule included in each microdroplet.
26. The method of any one of 1-25, wherein not more than 10 fg of the nucleic acid template molecule in encapsulated in the microdroplet.
27. The method of 26, wherein not more than 5 fg of the nucleic acid template molecule in encapsulated in the microdroplet.
28. The method of any one of 1-27, wherein the encapsulating and introducing occur in a single step.
29. A method for performing copy-number variation (CNV) analysis on a population of nucleic acids isolated from a biological sample, comprising:
   fragmenting the population of nucleic acids;
   encapsulating the fragmented population of nucleic acids in a plurality of microdroplets;
   introducing Multiple Displacement Amplification (MDA) reagents and a plurality of MDA primers, into each of the plurality of microdroplets;
   incubating the microdroplets under conditions effective for the production of MDA amplification products, wherein the incubating is effective to produce MDA amplification products from the nucleic acid template molecules;
   sequencing the MDA amplification products to determine the copy number of one or more nucleic acid sequences in the population of nucleic acids.
30. The method of 29, wherein the population of nucleic acids comprises genomic DNA.
31. The method of 29, wherein the genomic DNA is isolated from a single cell.
32. The method of 31, wherein the single cell is a cancer cell.
33. The method of 32, wherein the cancer cell is a circulating tumor cell (CTC).
34. The method of any one of 29-33, wherein each of the microdroplets, prior to the introducing and incubating steps, does not comprise more than one nucleic acid template molecule.
35. The method of any one of 29-34, wherein the MDA reagents comprise a Φ29 DNA or a Bst DNA polymerase.
36. The method of any one of 29-35, wherein the microdroplets have an internal volume of from about 0.001 picoliters to about 1000 picoliters.
37. The method of any one of 29-36, wherein each of the plurality of microdroplets comprises zero or one, and not more than one, nucleic acid template molecule.
38. The method of any one of 29-37, wherein the nucleic acid template molecule, MDA reagents, and MDA primers are loaded into a droplet dispenser to form the microdroplets.
39. The method of any one of 29-38, wherein one or more steps are performed under microfluidic control.
40. The method of any one of 29-38, wherein the microdroplets are generated via shaken emulsion.
41. The method of any one of 29-38, wherein the microdroplets are generated via microfluidic emulsion.
42. The method of any one of 29-41, wherein the fragmenting is via one of enzymatic fragmentation, heating, and sonication.
43. The method of any one of 29-41, wherein one or more cells of the biological sample are lysed to provide the nucleic acid template molecule.
44. The method of any one of 29-43, wherein the MDA amplification products for each microdroplet comprise a single MDA amplification product.
45. The method of any one of 29-43, wherein the MDA amplification products for each microdroplet comprise a plurality of different MDA amplification products.
46. The method of any one of 29-42 and 44-45, wherein the biological sample comprises one or more cells.
47. The method of 46, wherein the one or more cells comprises one or more circulating tumor cells (CTC).
48. The method of any one of 29-47, wherein the internal volume of each microdroplet is of an approximately equal volume.
49. The method of any one of 29-47, wherein the internal volume of each microdroplet is of a significantly different volume.
50. The method of any one of 29-49, wherein the number of microdroplets corresponds to the number of nucleic acid template molecules.
51. The method of any one of 29-49, wherein the number of nucleic acid template molecules to be amplified is varied by controlling the number of microdroplets generated.
52. The method of any one of 29-49, wherein the size of each microdroplet is varied in order to obtain a predetermined amount of MDA amplification product derived from the nucleic acid template molecule included in each microdroplet.
53. The method of any one of 29-52, wherein the encapsulating and introducing occur in a single step.
54. A composition comprising a microdroplet, comprising:
   a. a nucleic acid template molecule; and
   b. an MDA mixture, comprising:
      i. a plurality of MDA reagents comprising a polymerase enzyme capable of non-specifically amplifying the nucleic acid template molecule; and
      ii. a plurality of MDA primers.
55. The composition of 54, wherein the microdroplet does not comprise more than a single nucleic acid template molecule.
56. The composition of 54 or 55, wherein the microdroplet further comprises a detection component.
57. The composition of any one of 54-56, wherein the microdroplet further comprises one or more MDA amplification products produced from the nucleic acid template molecule.
58. The composition of any one of 54-57, wherein the microdroplet has an internal volume of from about 0.001 picoliters to about 1000 picoliters.
59. The composition of any one of 54-58, wherein the polymerase enzyme is Φ29 DNA polymerase or a Bst DNA polymerase.
60. The composition of any one of 54-59, wherein the MDA reagents comprise a magnesium reagent.
61. The composition of any one of 54-60, wherein the initial amount of the nucleic acid template molecule in the microdroplet is from about 0.001 pg to about 10 pg.
62. The composition of 61, wherein the initial amount of the nucleic acid template molecule in the microdroplet is from about 0.01 pg to about 1 pg.
63. The composition of 62, wherein the initial amount of the nucleic acid template molecule in the microdroplet is from about 0.1 pg to about 1 pg.
64. The composition of any one of 54-63, wherein the composition comprises a plurality of monodisperse microdroplets.
65. The composition of any one of 54-63, wherein the composition comprises a plurality of polydisperse microdroplets. 66. The composition of any one of 54 and 56-65, wherein the microdroplet comprises a plurality of nucleic acid template molecules. 67. The composition of any one of 54-60 and 64-66, wherein the microdroplet does not comprise more than 10 fg of the nucleic acid template molecule. 68. The composition of 67, wherein the microdroplet does not comprise more than 5 fg of the nucleic acid template molecule.

### EXAMPLES

As can be appreciated from the disclosure provided above, the present disclosure has a wide variety of applications. Accordingly, the following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Those of skill in the art will readily recognize a variety of noncritical parameters that could be changed or modified to yield essentially similar results. Thus, the following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for.

### Example 1: Preparation of Shaken Emulsion Droplets

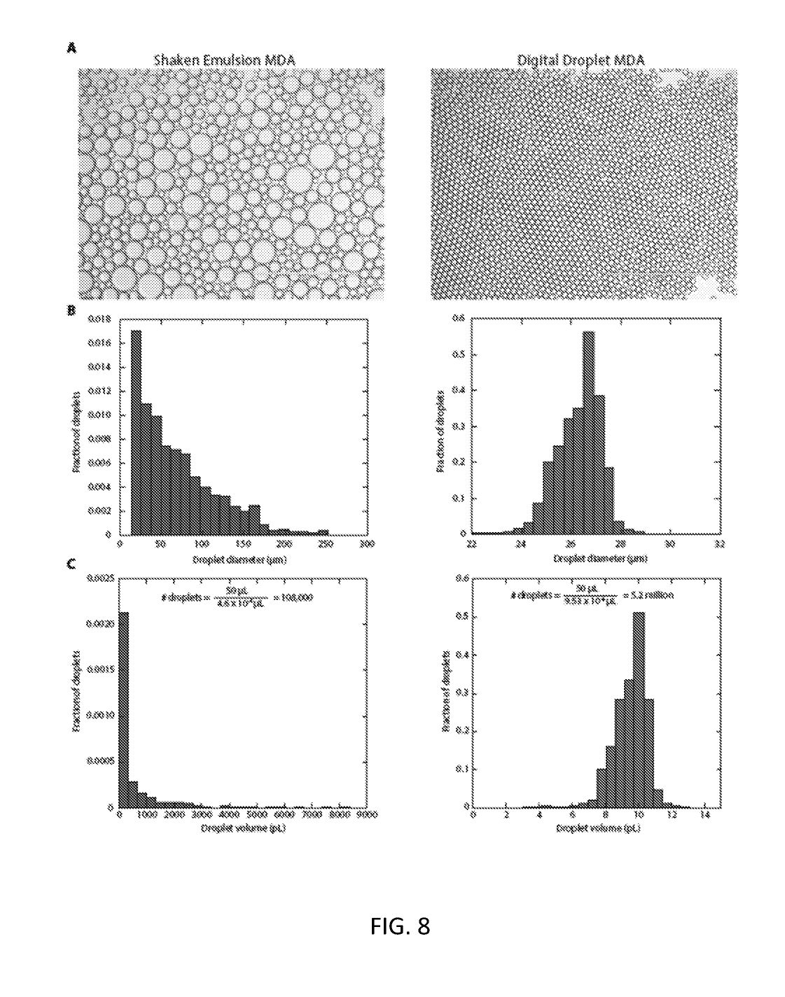

Shaken emulsions were generated by adding 30 μL of HFE-7500 fluorinated oil (3M, catalog no. 98-0212-2928-5) and 2% (w/w) PEG-PFPE amphiphilic block copolymer surfactant (RAN Technologies, catalog no. 008-FluoroSurfactant-1G) to 30 μL of MDA reaction mixture. Alternatively, HFE-7500 fluorinated oil with 2% PicoSurf1 (Dolomite Microfluidics) can be used. The combined mixture was vortexed at 3000 rpm for 10 seconds using a VWR vortexer, creating droplets ranging in diameter from 15 μm to 250 μm (FIG. 8). At the conclusion of incubation, 10 μL of perfluoro-1-octanol (Sigma Aldrich) was added, the mixture was vortexed to coalesce the droplets, and the aqueous layer was extracted with a pipette. A detailed protocol for shaken emulsion formation can be found in Example 7 below.

### Example 2: Preparation of Monodisperse Microfluidic Emulsion Droplets

The poly(dimethylsiloxane) (PDMS) microfluidic device used to generate monodisperse emulsions was fabricated by pouring uncured PDMS (10.5:1 polymer-to-crosslinker ratio) over a photolithographically-patterned layer of photoresist (SU-8 3025, MicroChem) on a silicon wafer (19). The device was cured in an 80° C. oven for 1 hr, extracted with a scalpel, and inlet ports were added using a 0.75 mm biopsy core (World Precision Instruments, catalog no. 504529). The device was bonded to a glass slide using O₂ plasma treatment and channels were treated with Aquapel (PPG Industries) to render them hydrophobic. Finally, the device was baked at 80° C. for 10 min. Commercial microfluidic droplet makers and pumps may also be used to generate monodisperse emulsions for the methods described herein, e.g., ddMDA.

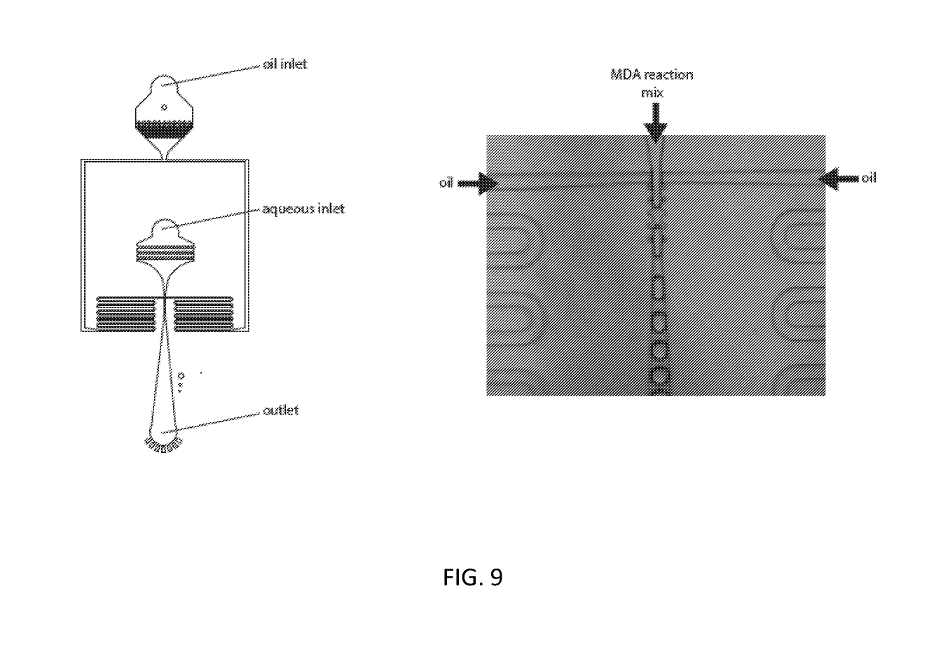

The MDA reaction mixture and HFE-7500 fluorinated oil with 2% (w/w) PEG-PFPE amphiphilic block copolymer surfactant (RAN Biotechnologies) were loaded into separate 1 mL syringes and injected at 300 and 500 μL/hr, respectively, into a flow-focusing droplet maker using syringe pumps (New Era, catalog no. NE-501) controlled with a custom Python script. Alternatively, HFE-7500 fluorinated oil with 2% PicoSurf1 (Dolomite Microfluidics) may also be usable and is available for purchase. The droplet maker generated monodisperse droplets ˜26 μm in diameter (See FIG. 8, Panels A-C), which were collected into a PCR tube. Droplets in this size range are stable during the ddMDA reaction. At the conclusion of incubation, 10 μL of perfluoro-1-octanol was added, the emulsion was vortexed to coalesce the droplets, and the aqueous layer was extracted with a pipette. A detailed protocol for microfluidic device fabrication and emulsification can be found Example 8 below. An example of a droplet maker which can be used to perform the methods described herein is illustrated in FIG. 9.

### Example 3: Extraction, Fragmentation, and Amplification of Genomic DNA

Purified E. coli K12(DH10B) cells were obtained from New England BioLabs (catalog no. C3019H), lysed, and purified using PureLink Genomic DNA Mini Kit (Life Technologies, catalog no. K1820-00). 10 kilobase fragments were gel-extracted following a 10-minute digestion with NEBNext dsDNA Fragmentase (NEB, catalog no. M0348S) of 800 ng DNA and quantified using a NanoDrop (Thermo Scientific). MDA reactions were performed using REPLI-g single cell kit (Qiagen, catalog no. 150343). Purified DNA (0.05 pg, 0.5 pg, and 5 pg) was incubated with 3 μL Buffer D2 and 3 μL H2O for 10 min at 65° C. After stopping by adding 3 μL stop solution, the reaction was divided in two and a master mix including nuclease-free H2O, REPLI-g Reaction Buffer, and REPLI-g DNA Polymerase was added to each partition. The MDA reactions were either incubated at 30° C. for 16 hrs in bulk or as an emulsion.

### Example 4: Single E. Coli Cell Sorting and Whole Genome Amplification

OneShot TOP10 chemically competent E. coli cells (Life Technologies, catalog no. C4040-10) were cultured in LB media for 12 hours, diluted in water, and stained with 0.25×SYBR Green I (Life Technologies, catalog no. S-7563). Following cell stain, the cell solution was imported into a BD FACS Aria II. Single positive events were sorted into 10 separate wells of a 96-well plate. 3 μL Buffer D2 and 3 μL H₂O were added to each well, after which the plate was heated at 98° C. for 4 minutes. This heat step lyses the cells and fragments the genomic DNA to adequate lengths for ddMDA (e.g., 5-15 kilobases). After heating, the reaction was stopped by adding 3 μL stop solution to each well. Next, master mix including nuclease-free H₂O, REPLI-g Reaction Buffer, and REPLI-g DNA Polymerase was added to each well. The MDA reactions were either incubated at 30° C. for 16 hrs in bulk or as an emulsion.

### Example 5: Digital Droplet PCR and MDA

Digital PCR and MDA experiments were performed with phage lambda genomic DNA as template (NEB, catalog no. N3011S). For digital PCR, the template was mixed in bulk with primers (IDT), TaqMan probe (IDT) and 2× Platinum Multiplex PCR Master Mix (Life Technologies, catalog no. 4464268) in a total volume of 100 μL. The sequences of the primers and probes were—Lambda Fwd: 5′-GCCCTTCTTCAGGGCTTAAT-3′ (SEQ ID NO:1); Lambda Rev: 5′CTCTGGCGGTGTTGACATAA-3′ (SEQ ID NO:2); Lambda Probe: 5′/6-FAM/ATACTGAGC/ZEN/ACATCAGCAGGACGC/3IABkFQ/-3′ (SEQ ID NO:3). Primers and probe were used at concentrations of 1 μM and 250 nM, respectively, and target a 110-basepair region in the lambda phage genome. Reaction mixture and HFE-7500 fluorinated oil with 2% (w/w) PEG-PFPE amphiphilic block copolymer surfactant were loaded into separate 1 mL syringes and injected at 300 and 600 μL/hr, respectively, into a flow-focusing device. After collecting the emulsion in PCR tubes, the oil underneath the emulsion was removed using a pipette and replaced with FC-40 fluorinated oil (Sigma-Aldrich, catalog no. 51142-49-5) with 5% (w/w) PEG-PFPE amphiphilic block copolymer surfactant. This oil/surfactant combination yields greater stability during the heated ddMDA reaction than the HFE oil combination. The emulsion was transferred to a T100 thermocycler (BioRad) and cycled with the following program: 95° C. for 2 min, followed by 35 cycles of 95° C. for 30 s, 60° C. for 90 s, and 72° C. for 20 s, followed by a final hold at 12° C.

For digital MDA, the template was mixed with reagents from the REPLI-g single cell kit as described previously, and combined with a DNA dye (EvaGreen, Biotium). The reaction mixture was emulsified through a flow-focusing device connected to syringes containing the reaction mixture and HFE-7500 fluorinated oil with 2% (w/w) PEG-PFPE amphiphilic block copolymer surfactant. The collected emulsion was incubated at 30° C. for 16 hrs. Since thermocycling is not required, FC-40 replacement is not necessary for digital MDA.

### Example 6: Library Prep and Sequencing Parameters

Bacterial libraries were prepared from 1 ng genomic DNA from each sample using the Nextera XT sample preparation kit (Illumina). The resulting libraries were quantified using a high sensitivity Bioanalyzer chip (Agilent), a Qubit Assay Kit (Invitrogen), and qPCR (Kapa Biosystems). Bacterial libraries varied between 800-1000 bp in fragment size. All libraries were pooled in equimolar proportions and sequenced using an Illumina MiSeq with 150 bp paired-end reads, and later using an Illumina HiSeq with 100 bp paired-end reads.

Sequencing data was mapped to the E. coli K12 DH10B reference genome using the BWA Whole Genome Sequencing program available on BaseSpace (Illumina). Mapped data was converted to SAM files and pileup files were generated using SAMtools. Genomic coverage as a function of genome position was determined by parsing the number of aligned reads from the pileup file, dividing each read number by the average read number, and consolidating the normalized data into 10,000 bp bins.

### Example 7: MDA in Shaken Emulsion Droplets

Example 7 provides one example of a method for generating shaken emulsion droplets for use in performing the methods and providing the compositions described herein.

Step 1: Immediately following preparation of 50 μl REPLI-g single cell reaction mixture (Qiagen, catalog no. 150343) in a PCR tube, add 50 μl of HFE-7500 fluorinated oil (3M, catalog no. 98-0212-2928-5) with 2% (w/w) PEG-perfluoropolyether amphiphilic block copolymer surfactant (RAN Biotechnologies, catalog no. 008-FluoroSurfactant-1G).

Step 2: Hold PCR tube containing 100 μl combined mixture horizontally on a VWR Vortexer 2 (VWR, catalog no. 58816-123). Vortex for 10 seconds at 3000 rpm.

Step 3: After vortexing hold PCR tube vertically. A white translucent emulsion should appear in the supernatant.

Step 4: Incubate PCR tube for 16 hours at 30° C.

Step 5: Following 16-hour incubation, heat PCR tube for 20 min at 70° C. to inactivate Φ29 DNA polymerase.

Step 6: Add 10 μl perfluoro-1-octanol (Sigma Aldrich, catalog no. 370533-5G) to supernatant. Pipet up and down vigorously and centrifuge briefly. This serves to destabilize the surfactant, thus coalescing the droplets.

Step 7: Extract supernatant from PCR tube. DO NOT extract any of the oil phase.

Step 8: Clean DNA using a DNA Clean and Concentrator-5 (Zymo Research, catalog no. D4004). Elute in 10 μl H2O.

### Example 8: ddMDA in Monodisperse Microfluidic Emulsion Droplets Fabricating PDMS Devices

Example 8 provides one example of a method for fabricating PDMS devices for use in performing microfluidic emulsion in connection with the methods and compositions described herein.

Step 1: Create a device master by spin-coating a 20 μm-thick layer of photoresist (SU-8 3025, Microchem) onto a silicon wafer, followed by patterned UV exposure and resist development (1).

Step 2: Combine 4 grams of Sylgard 184 Silicone Elastomer curing agent with 42 grams of Sylgard 184 Silicone Elastomer base (Dow Corning) in a plastic cup.

Step 3: Use an electric mixer to mix curing agent and base until mixture is white and bubbly.

Step 4: De-gas mixture by placing in a vacuum chamber for 20 min.

Step 5: Pour 30 grams of newly formed PDMS over previously made photolithographically patterned layer of photoresist on silicon wafer.

Step 6: Cure PDMS by placing in an 80° C. oven for 3 hr.

Step 7: Use a scalpel to cut out area of cured PDMS patterned by photoresist.

Step 8: Use a 0.75 mm biopsy core (World Precision Instruments, catalog no. 504529) to punch holes in the inlet and outlet ports of the device (denoted in FIG. 7 (left)).

Step 9: Wash device with isopropanol and air-dry.

Step 10: Bond device to a glass slide following a 30-second treatment of 1 mbar O₂ plasma in a 300 W plasma cleaner. Devices can also be bonded to tape (Thompson and Abate (2013) “Adhesive-based bonding technique for PDMS microfluidic devices.” Lab Chip, 13, 632-5).

Step 11: Place bonded device in 80° C. oven for 30 min.

Step 12: Using a syringe pre-loaded with Aquapel (PPG Industries) and connected to polyethylene micro tubing (Scientific Commodities, catalog no. BB31695-PE/2), flush all channels of device to make them hydrophobic.

Step 13: Place flushed device in 80° C. oven for an additional 10 min.

Step 14: Carefully inspect device using a microscope for presence of non-bonded or obstructed channels.

### Example 9: Generating Monodisperse Droplets

Example 9 describes one example of a method for generating monodisperse droplets for use in performing the methods and providing the compositions described herein.

Step 1: UV-treat the following for 30 min: polyethylene micro tubing, two 1 mL syringes, previously prepared microfluidic device (as described in Example 8), one PCR tube.

Step 2: Pre-load one UV-treated syringe with at least 200 μL HFE-7500 fluorinated oil with 2% (w/w) PEG-perfluoropolyether amphiphilic block copolymer surfactant.

Step 3: Pre-load second UV-treated syringe with 50 μL REPLI-g single cell reaction mixture back-filled with at least 200 μL HFE-7500 fluorinated oil to prevent bottoming out of syringe.

Step 4: Attach 8 inches of polyethylene micro tubing to syringe needles.

Step 5: Place syringes in two syringe pumps (New Era, catalog no. NE-501) connected to a computer controlled with a custom pump control program.

Step 6: Prime both syringes using the prime function in the pump control program.

Step 7: Attach polyethylene micro tubing connected to oil syringe to the “oil inlet” denoted in FIG. 7 (left).

Step 8: Attach polyethylene micro tubing connected to syringe with REPLI-g single cell reaction mixture to the “aqueous inlet” denoted in FIG. 7 (left).

Step 9: Attach one 4-inch piece of tubing to device outlet denoted in FIG. 7. Empty tubing into UV-treated PCR tube.

Step 10: Set flow rate of syringe with REPLI-g single cell reaction mixture at 300 μL/hour and flow rate of oil syringe at 500 μL/hour.

Step 11: Start flow program. Use a microscope to watch the formation of drops at the interface between oil and aqueous channels (see FIG. 7 image at right).

Step 12: Observe flow of droplets into outlet, through polyethylene micro tubing, and into PCR tube. Stop pump control program once entirety of 50 μL reaction mixture has been converted into droplets.

Step 13: Incubate PCR tube for 16 hours at 30° C.

Step 14: Following 16-hour incubation, heat PCR tube for 20 min at 70° C. to inactivate Φ29 DNA polymerase.

Step 15: Add 10 μL per fluoro-1-octanol to supernatant. Pipet up and down vigorously and centrifuge briefly. This serves to destabilize the surfactant, thus coalescing the droplets.

Step 16: Extract supernatant from PCR tube, where there is no extraction of the oil phase.

Step 17: Clean DNA using a DNA Clean and Concentrator-5. Elute in 10 μL H2O.

Digital Droplet MDA Workflow

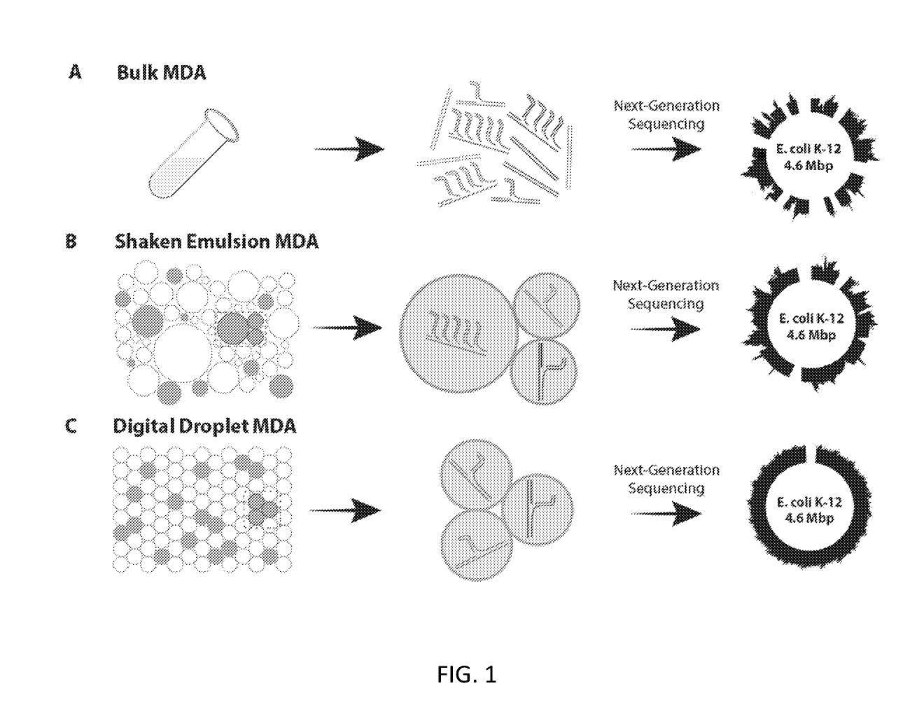

Referring to FIG. 1, Panels A-C illustrate various methods of amplifying E. coli nucleic acid template molecule(s) and then performing next-generation sequencing to determine the sequence of the nucleic acid template molecule(s).

Panel A illustrates amplifying nucleic acid template molecule(s) via bulk multiple displacement amplification (bulk MDA). Bulk MDA does not constrain the exponential nature of the reaction. Instead bulk MDA demonstrates sequence specific (i.e., bias) amplification, where specific sequences of nucleic acids template molecule(s) are amplified disproportionately relative to other sequences. As a result, the biased amplified sequences are amplified with higher coverage, while other sequences are amplified with lower coverage. Uneven coverage creates major challenges with sequencing, including inefficient use of sequencing reads, difficulty confidently assembling genomes using low-covered regions, and un-sequenced gaps in the genome (FIG. 1A, right).

Panel B illustrates amplifying nucleic acid template molecule(s) via shaken emulsion MDA. Because the isolated reactors are not physically connected to one another, the reactions occur independently and in parallel, allowing each compartment to amplify to saturation. Consequently, the representation of each template in the amplified product is far more uniform. Nevertheless, “shaken” emulsions consist of polydisperse droplets, in which the droplet volumes can vary by thousands of times. Because the number of product molecules at saturation scales with the volume of the reactor, reactor polydispersity can result in bias.

Panel C illustrates amplifying nucleic acid template molecule(s) via ddMDA in droplets of equal volume. Panel C illustrates that each nucleic acid template molecule is compartmentalized within a single microdroplet such that the single nucleic acid template molecule does not compete with other nucleic acids for resources (e.g., primers, reagents) in order to produce MDA amplification products. Instead because each single nucleic acid template molecule is allocated the same resources, each nucleic acid template molecule is uniformly amplified.

To ensure that single molecules are amplified the template concentration should be sufficiently low so that only a small percentage of droplets, typically <10%, contain a molecule, in accordance with Poisson statistics. This reduces the number of product molecules generated, but provides better uniformity (FIG. 1, Panel C, right). Moreover, since MDA is an extremely efficient reaction yielding copious DNA per unit volume, the small number of productive droplets provides more than enough material for sequencing.

Non-Specific Quantification of DNA with ddMDA

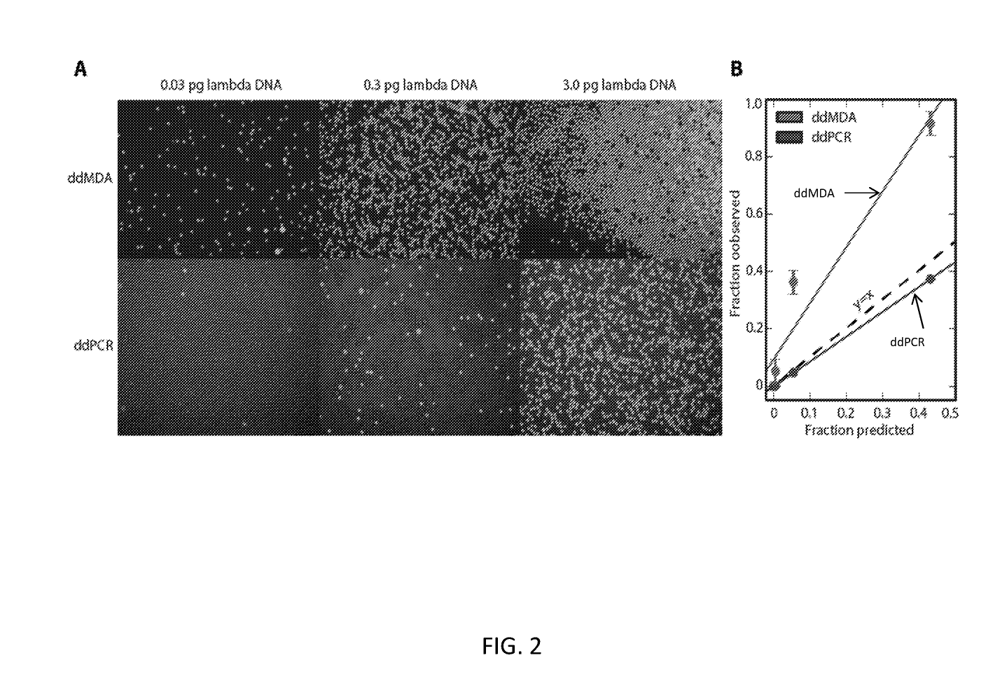

Digital droplet MDA enables uniform amplification of DNA by compartmentalizing and amplifying single template molecules in isolated droplet reactors. If a fluorescent reporter is included that indicates when a given droplet undergoes amplification, and thus contains a template molecule, it can also be used to quantify nucleic acids in solution by counting the fractions of fluorescent and dim droplets. This process is similar to digital droplet PCR (ddPCR), a more accurate alternative to qPCR for measuring DNA concentration, except that whereas ddPCR counts known templates, ddMDA quantitates any template amplifiable with the reaction, including templates of unknown sequence. To illustrate this, ddMDA was applied to quantify the concentrations of Lambda phage genomic fragments in solution, comparing the results with ddPCR (FIG. 2). The small Lambda phage genome offers a convenient source of DNA for quantification of amplification and contamination. Because ddPCR uses specific primers and probes, fewer fluorescent droplets are observed for the same concentration compared to ddMDA (FIG. 2, Panel A). In addition, the TaqMan probe required by ddPCR leads to higher background fluorescence than the non-specific dye used in ddMDA (FIG. 2, Panel A). Moreover, whereas the prediction based on Poisson encapsulation of single molecules is close to the ddPCR data (FIG. 2, Panel B), digital MDA systematically overestimates concentration (FIG. 2, Panel B). This can be rationalized by the specific nature of PCR versus the non-specific nature of MDA: Whereas ddPCR yields approximately one fluorescent droplet for each target genome in the sample, ddMDA does so for every genomic fragment amplifiable with the reaction. As the DNA concentration increases, the probability of multiple template molecules being encapsulated in the droplets increases too, leading to a larger fraction of droplets with two, three, or more molecules. Nevertheless, since this phenomenon is accounted for by the Poisson distribution, the method can still be used at these concentrations, although precision is reduced. Thus, fragmented or highly contaminated DNA will yield higher concentrations using ddMDA compared to ddPCR. This is important to the effectiveness of ddMDA non-specific DNA quantitation and for amplifying low-input DNA without regards to sequence.

Next Generation Sequencing of ddMDA-Amplified DNA

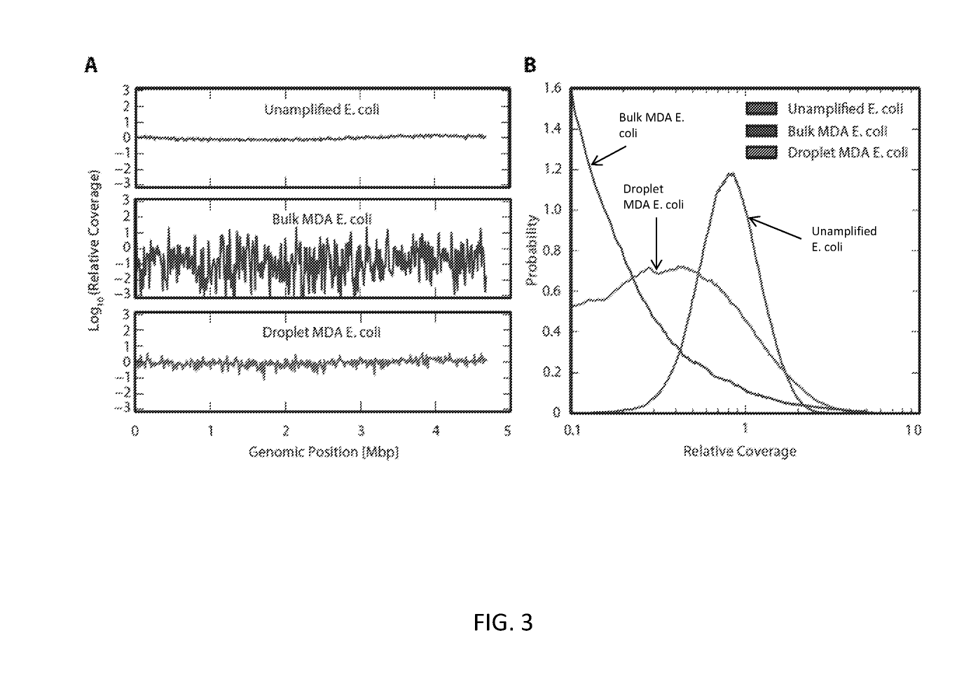

To investigate the effectiveness of ddMDA for amplifying low-input DNA for sequence analysis, samples prepared in different ways were sequenced and the results were compared: unamplified E. coli DNA (no amplification bias), E. coli DNA amplified using bulk MDA (the current standard), and E. coli DNA amplified using monodisperse ddMDA (the best-case scenario of compartmentalized reactions). E. coli genomes are used instead of Lambda phage genomes due to their greater size and complexity, thus offering greater applicability to next generation sequencing techniques. The starting concentration for the MDA reactions was 0.5 pg, corresponding to the genomes of ˜100 E. coli cells. The unamplified sample, not surprisingly, exhibited extremely uniform coverage with the exception of long-ranged systematic variation that may be representative of the bacteria's natural DNA replication cycle (FIG. 3, Panel A, top row). When the sample was subjected to bulk MDA, substantial amplification bias was observed causing significant over- and under-coverage of regions (FIG. 3, Panel A, middle row). In contrast, when the MDA amplification was constrained in monodisperse droplets, no subset of templates dominated the final product, resulting in uniform coverage across the genome (FIG. 3, Panel A, bottom row).

To further quantify the differences in sequencing bias for these preparation methods, the probability density of coverage levels for the three samples was plotted (FIG. 3, Panel B). Unamplified E. coli DNA had a narrow distribution, with little variation in coverage. In contrast, the coverage of DNA amplified by bulk MDA was extremely broad, with many regions exhibiting very low or very high coverage. This variation causes a number of challenges. The limited data for under-covered regions makes it challenging to assemble long sequences spanning these regions, since the low-coverage junctions cannot be determined with high confidence. Additionally, the high-coverage regions are wasteful of sequencing, since these regions are already covered adequately; they comprise a large fraction of sequencing data but offer little additional information. DNA amplified by ddMDA has a coverage distribution similar to the unamplified best-case scenario, but with larger bias. ddMDA thus yields amplified DNA that approaches the uniformity of unamplified material.

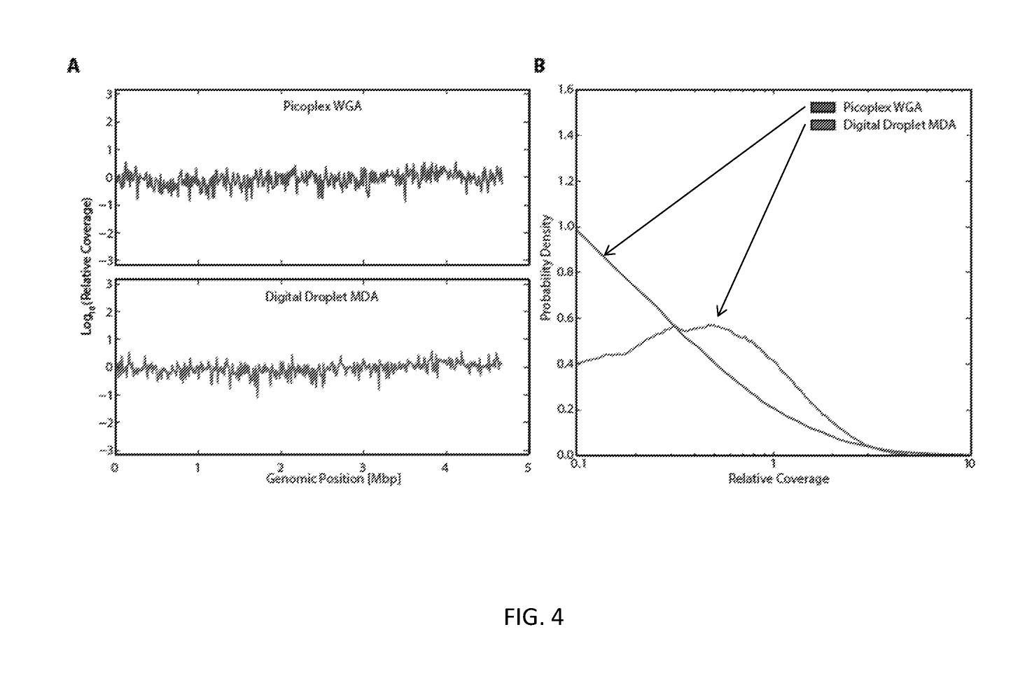

In order to further validate the utility of ddMDA as a reliable whole genome amplification method, sequenced DNA from a PCR-based WGA kit (PicoPLEX WGA, NEB) was compared to that of ddMDA. The PicoPLEX WGA kit lead to relatively even coverage, but still possessed a large fraction of under-covered reads (FIG. 4). This demonstrated the unique ability of ddMDA to yield minimal amplification bias.

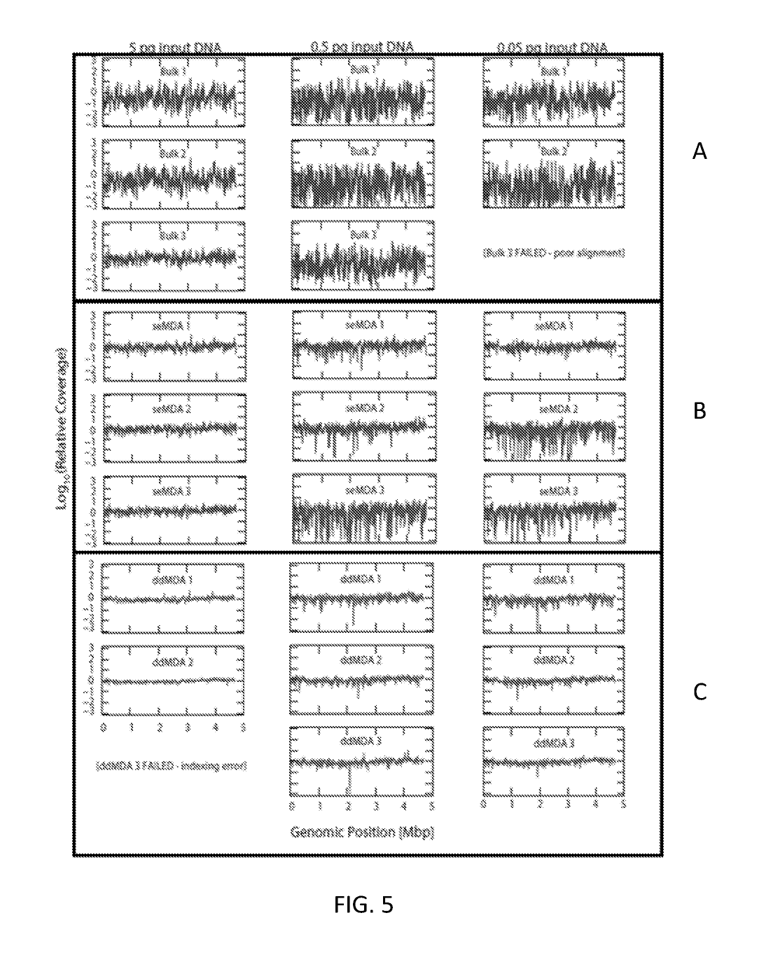

To further compare the differences in sequence bias obtained with the different methods of preparation, fresh samples were prepared using the three amplification methods described in FIG. 1 (bulk MDA, shaken emulsion MDA, and ddMDA) at three different input concentrations: 5 pg (˜1000 genomes), 0.5 pg (˜100 genomes) and 0.05 pg (˜10 genomes). Next-generation sequencing of these samples reveals that, indeed, bulk MDA yields poor uniformity in sequencing coverage, while ddMDA and shaken emulsion MDA exhibit significantly improved uniformity (FIG. 5).

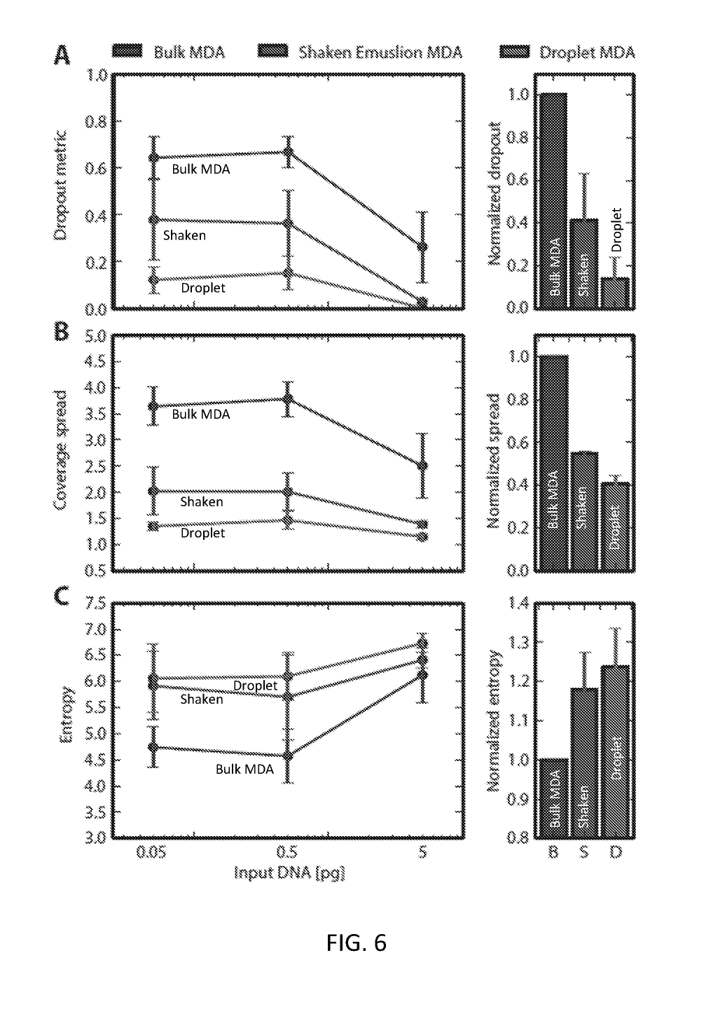

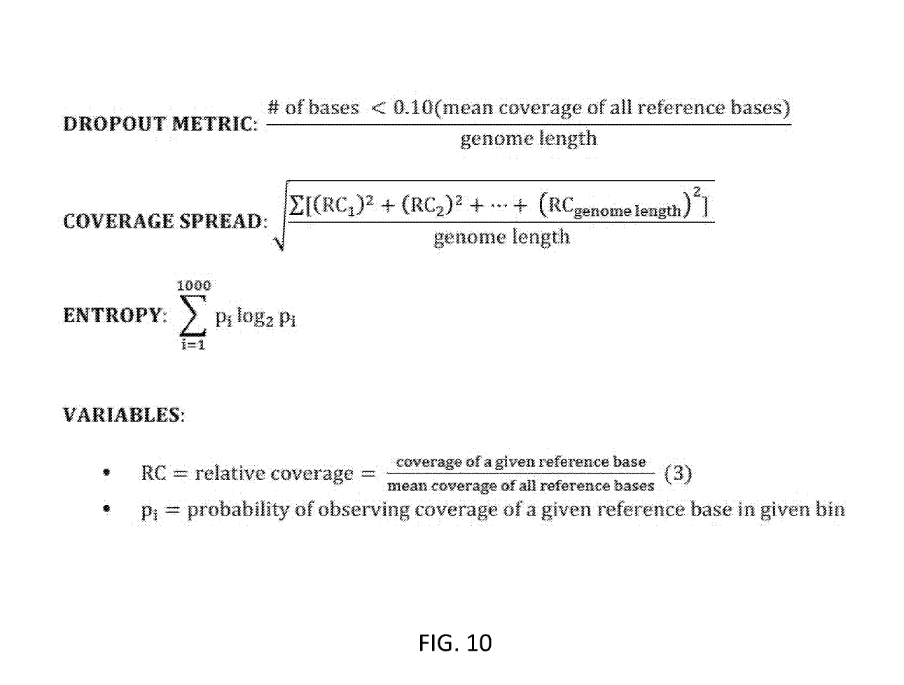

Next-generation sequencing of unknown genomes necessitates near-complete coverage of all regions. However, amplification can result in biased genomic representation, in which low-abundance regions may not be sufficiently covered during sequencing. To quantify the frequency of this occurrence for the different preparation methods and concentrations, a dropout metric was utilized that represents the number of genomic regions that are significantly under-covered (FIG. 6, Panel A). Specifically, the fraction of bases covered at less than 10% of the mean coverage for each sample was analyzed (the equation used can be found in FIG. 10). In the bulk MDA samples, a significant fraction of the genome was not detected for low and moderate input concentrations (FIG. 6, Panel A). For higher input concentrations, the fraction of under-coverage was lower, but still significant. In the shaken emulsion MDA samples, compartmentalization resulted in a marked reduction of dropout for all three concentrations (FIG. 6, Panel A); however, substantial dropout was still observed. The ddMDA samples further reduced the number of dropout regions and maintained low dropout even down to 10 genome equivalents of E. coli DNA (FIG. 6, Panel A). This trend in which bulk MDA results in the worst data and ddMDA the best is evident when all three concentrations are normalized to the bulk preparation and averaged (FIG. 6, Panel A, bottom panel).

Another important factor in sequencing low-input DNA is the efficiency of sequencing—specifically, ensuring that each additional read that is sequenced provides maximum new information content. If significant coverage spread exists, small, highly covered regions can comprise a large fraction of the sequenced reads, thus requiring increased sequencing expenditure to observe the low-covered regions. To quantify this disparity in coverage, a metric was used that estimates coverage spread, calculated as the root mean square of the relative coverage (FIG. 6, Panel B). The equation used can be found in FIG. 10. The trend between samples is similar to the trend in the dropout metric, since regions that are under-covered also tend to drop out, and is also evident when the points are normalized to the bulk results and averaged (FIG. 6, Panel B, bottom panel). This shows that compartmentalized MDA significantly reduces coverage disparity, maximizing the useful information content in the reads that are obtained and, consequently, allowing an equal amount of new information to be obtained with less total sequence expenditure compared to bulk MDA.

Another valuable metric for estimating uniformity of coverage and the likelihood of being able to generate an accurate assembly is the informational entropy, a measurement used to estimate the randomness of a signal, such as the coverage signals obtained from FIG. 3, Panel A. When sequencing unknown genomes, high entropy representing a coverage distribution that is maximally randomized over the entire sequence is ideal. The informational entropies are similar for ddMDA and shaken emulsion MDA, and both perform better than bulk MDA (FIG. 6, Panel C). As before, the trend is present when normalizing and averaging over input concentrations (FIG. 6, Panel C, right panel). The equation used for informational entropy can be found in FIG. 10. These data demonstrate that compartmentalized ddMDA is an effective means to maximally cover the genome with minimal sequencing expenditure.

Next Generation Sequencing of ddMDA-Amplified DNA from Single Cells

In order to further demonstrate the utility of ddMDA for whole genome amplification, the methodology was applied to single E. coli cells. Amplifying single cells is of enormous importance for single cell analysis, in particular the study of single uncultivable microbes and individual cancer cells. Though valuable, the procedure is much more complex than amplifying purified DNA. The single cell must generally be reliably lysed and fragmented into molecules compatible with MDA. Furthermore, a number of precautions, including UV exposure and sterile procedures, must be taken to minimize contamination and DNA loss, which become especially problematic with such small amounts of starting material. In this work, single E. coli cells were FACS-sorted into individual wells, lysed and heat-fragmented, and the MDA reactions were emulsified using the identical procedure from before. In particular, 2 different cells amplified with ddMDA were compared to 2 different cells amplified with standard bulk MDA. After sequencing the samples and performing the identical bioinformatic analyses described previously, significant amounts of amplification bias were found in the cells amplified by bulk MDA (FIG. 7, Panel A, top left panel). In particular, Bulk MDA Cell 2 possessed a massive amount of under-amplification, yielding complete dropout of several 10,000 basepair regions (denoted by gaps in the coverage plot). The two cells amplified by ddMDA, on the other hand, had significantly more uniform coverage (FIG. 7, Panel A, top right panel). These results are further validated by analyzing the probability density of the four samples (FIG. 7, Panel B). Though contamination and DNA loss are a concern, the dramatic difference in coverage between bulk MDA and ddMDA demonstrate the adaptability of this technique to single bacterial cells.

What is claimed is:

1. A method of non-specifically amplifying a nucleic acid template molecule, the method comprising:
encapsulating in a microdroplet a nucleic acid template molecule obtained from a biological sample;
introducing Multiple Displacement Amplification (MDA) reagents and a plurality of MDA primers into the microdroplet; and
incubating the microdroplet under conditions effective for the production of MDA amplification products, wherein the incubating is effective to produce MDA amplification products from the nucleic acid template molecule.

2. The method of claim 1, wherein the microdroplet, prior to the introducing and incubating steps, does not include more than one nucleic acid template molecule.

3. The method of claim 1 or 2, wherein the MDA reagents comprise a Φ29 DNA polymerase.

4. The method of any one of claims 1-3, wherein the microdroplet has an internal volume of from about 0.001 picoliters to about 1000 picoliters.

5. The method of any one of claims 1-4, wherein the encapsulating comprises encapsulating in a plurality of microdroplets a plurality of nucleic acid template molecules obtained from one or more biological samples, the introducing comprises introducing MDA reagents and a plurality of MDA primers into each of the plurality of microdroplets, and the incubating comprises incubating the plurality of microdroplets under conditions effective for the production of MDA amplification products, wherein the incubating is effective to produce MDA amplification products from the nucleic acid template molecules.

6. The method of claim 5, wherein each of the plurality of microdroplets comprises zero or one, and not more than one, nucleic acid template molecule.

7. The method of any one of claims 1-6, wherein the nucleic acid template molecule, MDA reagents, and MDA primers are loaded into a droplet dispenser to form the microdroplet.

8. The method of any one of claims 1-7, wherein one or more steps are performed under microfluidic control.

9. The method of any one of claims 1-7, wherein the microdroplet is generated via shaken emulsion.

10. The method of any one of claims 1-7, wherein the microdroplet is generated via microfluidic emulsion.

11. The method of any one of claims 1-10, wherein one or more nucleic acids of the biological sample are fragmented to provide the nucleic acid template molecule.

12. The method of claim 11, wherein the fragmentation is via one of enzymatic fragmentation, heating, and sonication.

13. The method of any one of claims 1-10, wherein one or more cells of the biological sample are lysed to provide the nucleic acid template molecule.

14. The method of any one of claims 1-13, further comprising determining the sequence of the MDA amplification products via next-generating sequencing (NGS).

15. The method of any one of claims 1-14, wherein the MDA amplification products comprise a single MDA amplification product.

16. The method of any one of claims 1-14, wherein the MDA amplification products comprise a plurality of different MDA amplification products.

17. The method of any one of claims 1-12 and 14-16, wherein the biological sample comprises one or more cells.

18. The method of claim 17, wherein the one or more cells comprises one or more circulating tumor cells (CTC).

19. The method of any one of claims 1-18, further comprising a step of introducing a detection component into each microdroplet, wherein detection of the detection component indicates the presence of one more MDA amplification products.

20. The method of claim 19, wherein the detection component is detected based on a change in fluorescence.

21. The method of claim 5, wherein the internal volume of each microdroplet is of an approximately equal volume.

22. The method of claim 5, wherein the internal volume of each microdroplet is of a significantly different volume.

23. The method of claim 5, wherein the number of microdroplets corresponds to the number of nucleic acid template molecules.

24. The method of claim 5, wherein the number of nucleic acid template molecules to be amplified is varied by controlling the number of microdroplets generated.

25. The method of claim 5, wherein the size of each microdroplet is varied in order to obtain a predetermined amount of MDA amplification product derived from the nucleic acid template molecule included in each microdroplet.

26. The method of any one of claims 1-25, wherein not more than 10 fg of the nucleic acid template molecule in encapsulated in the microdroplet.

27. The method of claim 26, wherein not more than 5 fg of the nucleic acid template molecule in encapsulated in the microdroplet.

28. The method of any one of claims 1-27, wherein the encapsulating and introducing occur in a single step.

29. A method for performing copy-number variation (CNV) analysis on a population of nucleic acids isolated from a biological sample, comprising:
fragmenting the population of nucleic acids;
encapsulating the fragmented population of nucleic acids in a plurality of microdroplets;
introducing Multiple Displacement Amplification (MDA) reagents and a plurality of MDA primers, into each of the plurality of microdroplets;
incubating the microdroplets under conditions effective for the production of MDA amplification products, wherein the incubating is effective to produce MDA amplification products from the nucleic acid template molecules;
sequencing the MDA amplification products to determine the copy number of one or more nucleic acid sequences in the population of nucleic acids.

30. The method of claim 29, wherein the population of nucleic acids comprises genomic DNA.

31. The method of claim 29, wherein the genomic DNA is isolated from a single cell.

32. The method of claim 31, wherein the single cell is a cancer cell.

33. The method of claim 32, wherein the cancer cell is a circulating tumor cell (CTC).

34. The method of claim any one of claims 29-33, wherein each of the microdroplets, prior to the introducing and incubating steps, does not comprise more than one nucleic acid template molecule.

35. The method of any one of claims 29-34, wherein the MDA reagents comprise a Φ29 DNA polymerase.

36. The method of any one of claims 29-35, wherein the microdroplets have an internal volume of from about 0.001 picoliters to about 1000 picoliters.

37. The method of any one of claims 29-36, wherein each of the plurality of microdroplets comprises zero or one, and not more than one, nucleic acid template molecule.

38. The method of any one of claims 29-37, wherein the nucleic acid template molecule, MDA reagents, and MDA primers are loaded into a droplet dispenser to form the microdroplets.

39. The method of any one of claims 29-38, wherein one or more steps are performed under microfluidic control.

40. The method of any one of claims 29-38, wherein the microdroplets are generated via shaken emulsion.

41. The method of any one of claims 29-38, wherein the microdroplets are generated via microfluidic emulsion.

42. The method of claim any one of claims 29-41, wherein the fragmenting is via one of enzymatic fragmentation, heating, and sonication.

43. The method of any one of claims 29-41, wherein one or more cells of the biological sample are lysed to provide the nucleic acid template molecule.

44. The method of any one of claims 29-43, wherein the MDA amplification products for each microdroplet comprise a single MDA amplification product.

45. The method of any one of claims 29-43, wherein the MDA amplification products for each microdroplet comprise a plurality of different MDA amplification products.

46. The method of any one of claims 29-42 and 44-45, wherein the biological sample comprises one or more cells.

47. The method of claim 46, wherein the one or more cells comprises one or more circulating tumor cells (CTC).

48. The method of any one of claims 29-47, wherein the internal volume of each microdroplet is of an approximately equal volume.

49. The method of any one of claims 29-47, wherein the internal volume of each microdroplet is of a significantly different volume.

50. The method of any one of claims 29-49, wherein the number of microdroplets corresponds to the number of nucleic acid template molecules.

51. The method of any one of claims 29-49, wherein the number of nucleic acid template molecules to be amplified is varied by controlling the number of microdroplets generated.

52. The method of any one of claims 29-49, wherein the size of each microdroplet is varied in order to obtain a predetermined amount of MDA amplification product derived from the nucleic acid template molecule included in each microdroplet.

53. The method of any one of claims 29-52, wherein the encapsulating and introducing occur in a single step.

54. A composition comprising a microdroplet, comprising:
a. a nucleic acid template molecule; and
b. an MDA mixture, comprising:
i. a plurality of MDA reagents comprising a polymerase enzyme capable of non-specifically amplifying the nucleic acid template molecule; and
ii. a plurality of MDA primers.

55. The composition of claim 54, wherein the microdroplet does not comprise more than a single nucleic acid template molecule.

56. The composition of claim 54 or claim 55, wherein the microdroplet further comprises a detection component.

57. The composition of any one of claims 54-56, wherein the microdroplet further comprises one or more MDA amplification products produced from the nucleic acid template molecule.

58. The composition of any one of claims 54-57, wherein the microdroplet has an internal volume of from about 0.001 picoliters to about 1000 picoliters.

59. The composition of any one of claims 54-58, wherein the polymerase enzyme is Φ29 DNA polymerase.

60. The composition of any one of claims 54-59, wherein the MDA reagents comprise a magnesium reagent.

61. The composition of any one of claims 54-60, wherein the initial amount of the nucleic acid template molecule in the microdroplet is from about 0.001 pg to about 10 pg.

62. The composition of claim 61, wherein the initial amount of the nucleic acid template molecule in the microdroplet is from about 0.01 pg to about 1 pg.

63. The composition of claim 62, wherein the initial amount of the nucleic acid template molecule in the microdroplet is from about 0.1 pg to about 1 pg.

64. The composition of any one of claims 54-63, wherein the composition comprises a plurality of monodisperse microdroplets.

65. The composition of any one of claims 54-63, wherein the composition comprises a plurality of polydisperse microdroplets.

66. The composition of any one of claims 54 and 56-65, wherein the microdroplet comprises a plurality of nucleic acid template molecules.

67. The composition of any one of claims 54-60 and 64-66, wherein the microdroplet does not comprise more than 10 fg of the nucleic acid template molecule.

68. The composition of claim 67, wherein the microdroplet does not comprise more than 5 fg of the nucleic acid template molecule.


SEQUENCE LISTING (3 sequences)

SEQ ID NO: 1 - DNA, 20 bp, Artificial Sequence
  synthetic oligonucleotide
gcccttcttcagggcttaat

SEQ ID NO: 2 - DNA, 20 bp, artificial sequence
  synthetic oligonucleotide
ctctggcggtgttgacataa

SEQ ID NO: 3 - DNA, 24 bp, Artificial Sequence
  synthetic oligonucleotide
atactgagcacatcagcaggacgc